(19) United States
(12) Patent Application Publication
(10) Pub. No.: US 2024/0069031 A1
(43) Pub. Date: Feb. 29, 2024

(54) SINGLE CELL SPATIAL METABOLOMICS FOR MULTIPLEXED CHEMICAL ANALYSIS

(71) Applicant: Georgia Tech Research Corporation, Atlanta, GA (US)

(72) Inventors: Ahmet F. Coskun, Atlanta, GA (US); Thomas Hu, Atlanta, GA (US)

(21) Appl. No.: 18/456,768

(22) Filed: Aug. 28, 2023

Related U.S. Application Data
(60) Provisional application No. 63/401,294, filed on Aug. 26, 2022.

(51) Int. Cl. G01N 33/58; C12M 3/06

(52) U.S. Cl. CPC G01N 33/58; C12M 23/16; G01N 2570/00

(57) ABSTRACT

Provided is a method of detecting analytes in a cell or tissue sample, the method comprising: a) introducing into the cell or tissue sample at least one tagging moiety, wherein the tagging moieties can interact with specific proteins of interest; b) detecting analytes and tagging moieties in the cell or tissue sample; c) spatially detecting proteins in the cell or tissue sample; and d) constructing a map of the analytes in the cell or tissue sample based on the data from steps b) and c). Also provided is a microfluidic chip using the method of detecting analytes, and methods of monitoring an in situ model of a tumor, methods of detecting cancer, and methods of determining response of a subject to a treatment protocol using the microfluidic chip.

### BACKGROUND

Metabolism is a set of chemical reactions that regulate cell function and response. Recent advances in metabolite profiling techniques have identified thousands of compounds in cells and tissues for applications in various cancers. Mass spectrometry has revealed key molecular networks that are upregulated and downregulated in multiple cell types. Mass spectrometry imaging (MSI) has also provided localization of metabolites and roles in diseases. However, the spatial resolution of these MSI methods has been limited to 20-100 μm, making it challenging to link single cells to the metabolic profiles. Another spatial metabolic profiling method, time-of-flight secondary ion mass spectrometry (TOF-SIMS) has allowed submicron metabolite mapping in cells and tissues, but the association of specific cell types to the metabolic profiles has been lacking. While a recent method, SpaceM,¹ performed correlations of matrix-assisted laser desorption/ionization (MALDI) based metabolic maps to the cultured cells, no technique can achieve cell-type and metabolic analysis in densely packed single cells within native tissues.

While significant progress has been made in time-dependent (T) metabolite networks and modeling, the location information (X, Y, and Z coordinates) of single metabolites and their spatial gradients have been lacking in the systems biology view of metabolism. Thus, there is an important need for four-dimensional (4D) metabolic analysis of cells to measure and model spatial coordinates (X-Y-Z) and time (T) or sample condition (S) of chemical networks in health and disease. These needs and others are at least partially satisfied by the present disclosure.

### SUMMARY

The present invention relates to a method to decipher spatially resolved metabolic regulation of immune cells and cancer cells in tumors at the single-cell level by incorporating untargeted spatial metabolomics and targeted multiplexed protein imaging in a single platform. This invention combines three-dimensional (3D) spatially resolved metabolic profiling framework (3D-SMF, Science Advances 2021) and multiplexed protein imaging method, imaging mass cytometry (IMC, Communications Biology 2021), to localize twenty individual cell types and determine the maps of hundreds metabolic marker in each cell type within native tissues from tumor biopsies and bio-inspired tumor chips.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF DRAWINGS

FIGS. 1A-1C depict the ScSpaMet pipeline for metabolite and protein profiling at the single-cell resolution. FIG. 1A shows an overview of ScSpaMet. Tissue samples on glass slides are labeled with metal-isotope conjugated antibodies followed by metabolic profiling with 3D-SMF and finally performing proteomic profiling using IMC. Created with Biorender.com FIG. 1B shows examples of ScSpaMet generating data in lung cancer tissue data. Left to right: PO3-channel ion, multiplex metabolites data pixel clustering, IMC imaging Histone H3 marker, multiplex IMC data overlay with pseudo coloring, virtual reconstructed H & E staining from IMC multiplex data. Scale bar 100 μm FIG. 1C shows examples of ScSpaMet generating data in Tonsil data. Left to right: PO3-channel ion, multiplex metabolites data pixel clustering, IMC imaging Intercalator marker, multiplex IMC data overlay with pseudo coloring, virtual reconstructed H & E staining from IMC multiplex data. Scale bar 100 μm

FIGS. 2A-2C depict the single-cell cross-modality registration pipeline. FIG. 2A shows single-cell protein-metabolite bi-modal registration pipeline. Input images consist of 3D-SMF and IMC-generated images. First, a template matching algorithm is used to find the corresponding matching region between the 3D-SMF region (smaller) inside the IMC region (larger). Next, the rotation offset between the two aligned and cropped images is calculated. Finally, the affine transformation of the two images is found to obtain bi-modal matched images. FIG. 2B shows registration result comparison between random, rotation registration, and affine registration. Left: Examples of the three registration methods between IMC (gray) and 3D-SMF (red) images and their corresponding inset. Scale bar 100 μm. FIG. 2C shows a comparison of Structure similarity and Normalized Root Mean Square Error between the three registration methods. Mann-Whitney-Wilcoxon test two-sided with Bonferroni correction (ns: 0.05<p, *: 0.01<p<=0.05, **: 0.001<p<=0.01, ***: 0.0001<p<=0.001, ****: p<=0.0001).

FIGS. 3A-3B depict metabolomic and proteomic modalities data analysis. FIG. 3A shows an overview of metabolomic and proteomic data generated by ScSpaMet. Tissue samples on glass slides are labeled with metal-isotope conjugated antibodies followed by metabolic profiling with TOF-SIMS and sequential proteomic profiling using IMC. The resulting outputs are n cells with distinct p_m metabolomic feature and p_p proteomic feature. The modalities have distinct feature spaces due to differences in feature number and variability across datasets. Created with Biorender.com. FIG. 3B shows examples of existing spatial-omics data integration pipelines show (i) same-modality integration, (ii) cross-modality integration with shared features, (iii) cross-modality multi-modal on the same cell. (i) is applied to remove the batch effect from both metabolite and protein datasets. The scSpaMet data didn't contain any share features (marker) or cell types. Created with Biorender.com

FIGS. 4A-4F depict how ScSpaMet identifies metabolite differences between tumor and stromal regions in human lung cancer tissues. FIG. 4A shows unsupervised single cell clusters from protein profiles in human lung cancer tissues. Created with Biorender.com. FIG. 4B shows a spatial projection of cell corresponding clusters from FIG. 4A. FIG. 4C shows: Left: definition of stroma and tumor region in tissue sample based on single cell phenotypes from FIG. 4A. Right: most expressed metabolite channels in stroma and tumor region from the differential analysis. FIG. 4D shows a comparison of single cell metabolites expression level for identified metabolite channels. Left: metabolite channels related to Glucose pathway and Cholesterol fragments. Middle: Metabolite channels related to amino acid fragments. Right: Bar graph of selected metabolite channels. Mann-Whitney-Wilcoxon test was two-sided with Bonferroni correction (ns: 0.05<p, ****: p<=0.0001). FIG. 4E shows metabolite channels related to identified lipid channels fragmentation. FIG. 4F shows a spatial projection of single cell metabolite expression level for selected metabolite channels.

FIGS. 5A-5D depict how ScSpaMet quantifies local metabolite competition in lung cancer as a function of endothelial cells. FIG. 5A shows a representative schematic showing the definition of distance to CD31+ cells in lung cancer tissues. Created with Biorender.com. FIG. 5B shows a Pearson correlation of metabolites channels intensity compared to single cell distance to CD31+ cells. Left: Heatmap showing Pearson correlation of selected metabolite channels compared to the distance to CD31+ cells. Right: Single cell spatial distance map to CD31+ cells in lung cancer tissues. FIG. 5C shows local metabolites competition as a distance of CD31+ cells between T-cells and tumor cells. Left: Selected metabolite channels showing positive and negative correlation of tumor cells (top) and T-cells (bottom). Right: spatial projection of T-cells and tumor cells' local metabolites competition for 48 m/z. FIG. 5D shows local metabolites competition as a distance of CD31+ cells between CD68 positive cells and tumor cells. Left: Selected metabolite channels showing positive and negative correlation of tumor cells (top) and T-cells (bottom). Right: spatial projection of T-cells and tumor cells' local metabolites competition for 74 m/z.

FIGS. 6A-6D depict how ScSpaMet identifies spatial signature of joint protein-metabolite signatures in lung cancer tissues across patients. FIG. 6A shows an overview of spatial joint protein-metabolite signatures identification using the scSpaMet pipeline. Using VAE joint embedding, cells are clustered based on their joint protein-metabolite profiles. A neighborhood graph is constructed based on cell spatial location. The corresponding cell neighborhood cell type frequencies are used to determine spatial joint protein-metabolite signature across patients. Created with Biorender.com. FIG. 6B shows a count of corresponding spatial joint protein-metabolite signatures across imaging regions in all the patients in lung cancer tissues. FIG. 6C shows the frequency of corresponding spatial joint protein-metabolite signatures across patients in lung cancer tissues. FIG. 6D shows a spatial projection of cell-corresponding spatial joint protein-metabolite signatures from FIG. 6A.

FIGS. 7A-7F depict how ScSpaMet identifies metabolite differences of B cell follicles regions in human tonsil tissues. FIG. 7A shows unsupervised single cell clusters from protein profiles in human tonsil tissues. Created with Biorender.com. FIG. 7B shows a spatial projection of cell corresponding clusters from FIG. 7A. FIG. 7C shows top expressed metabolite channels in regions inside and outside germinal centers across tonsil tissues from the differential analysis. FIG. 7D shows a comparison of single cell metabolites expression level for identified metabolite channels. Left: metabolite channels related to Glucose pathway and Cholesterol fragments. Middle: Metabolite channels related to amino acid fragments. Right: Bar graph of selected metabolite channels. Mann-Whitney-Wilcoxon test was two-sided with Bonferroni correction (****: p<=0.0001). FIG. 7E shows metabolite channels related to identified lipid channels fragmentation. FIG. 7F shows a spatial projection of single-cell metabolite expression level for selected metabolite channels.

FIGS. 8A-8C depict how ScSpaMet quantifies cell type-specific local metabolite competition in germinal centers. FIG. 8A shows a representative schematic showing the definition of local cell metabolite competition in human tonsil germinal center regions. Created with Biorender.com. FIG. 8B shows local competition of metabolites between B cells and FDCs (top), B cells and TFHs (middle), B cells in LZ with DZ (bottom). FIG. 8C shows a comparison of selected metabolite channels between GC LZ and GZ DZ.

FIGS. 9A-9D depict how ScSpaMet infers metabolites trajectory for B cell differentiation inside of germinal centers. FIG. 9A shows a representative schematic showing the definition of germinal center B cell trajectories. Created with Biorender.com. FIG. 9B shows B cell trajectories in the germinal center from protein markers in scSpaMet. Left: Unsupervised clustering of B cell protein markers and corresponding TSNE plot. Middle: Pseudotime analysis of B cell protein phenotype. Right: Identified B cell trajectories from single-cell phenotype. (1) the GC DZ B-cells to GC LZ B-cells and (2) the GC DZ B-cells to the activated B-cells. FIG. 9C shows an identified germinal center dark zone to light zone trajectory. FIG. 9D shows a spatial plot of germinal center dark zone to light zone trajectory (left) and germinal center dark zone to Activated B cells trajectory (right).

FIGS. 10A-10F depict how ScSpaMet characterizes proteins and metabolites in human endometrium samples. FIG. 10A shows unsupervised single cell clusters from proteins profiles in human endometrium tissues. Created with Biorender.com. FIG. 10B shows a spatial projection of cell corresponding clusters from FIG. 10A. FIG. 10C shows top expressed metabolite channels in identified cell types across endometrium tissues from the differential analysis. FIG. 10D shows a comparison of single cell metabolites expression level for identified metabolite channels. Left: metabolite channels related to Glucose pathway and Cholesterol fragments. Middle: Metabolite channels related to amino acid fragments. Right: Bar graph of selected metabolite channels. Mann-Whitney-Wilcoxon test was two-sided with Bonferroni correction (****: p<=0.0001). FIG. 10E shows metabolite channels related to identified lipid channels fragmentation. FIG. 10F shows a spatial projection of single-cell metabolite expression level for selected metabolite channels in obese (top) and lean (bottom) tissues.

FIGS. 11A-11D depict ScSpaMet analysis modules. FIG. 11A shows an overview of scSpaMet data analysis pipeline. Tissue samples on glass slides are labeled with metal-isotope conjugated antibodies and adjacent tissues stained with H & E are used for the identification of imaging regions for scSpaMet. This is followed by metabolic profiling with 3D-SMF and proteomic profiling using IMC. After imaging, single-cell registration is performed with downstream analysis. FIG. 11B shows an overview of ScSpaMet local metabolomic competition analysis pipeline. A single cell competes with their neighboring cell for metabolomic resources. ScSpaMet quantifies neighboring cell types' local metabolite competition. FIG. 11C shows an overview of ScSpaMet single cell joint modality VAE embedding pipeline. Single cell metabolomic and proteomic profiles are used as input for the VAE to extract joint embedding space representation. Joint embedding is used to decipher cell type metabolic states and enrichment as well as stratify patients. FIG. 11D shows an overview of ScSpaMet pseudo time analysis pipeline. Using single cell embedding, cell trajectory differentiation pseudotime analysis is used to measure metabolomic and proteomic change along trajectories.

FIGS. 12A-12B depict pixel clustering for SIMS data in tonsil tissues (FIG. 12A) and endometrium tissues (FIG. 12B). Scale bar 100 μm.

FIG. 13 depicts single-cell metabolite and protein profile correlation in lung cancer tissues. Each heatmap shows the correlation of metabolite channels with corresponding protein markers. Left to right shows ascending single-cell protein intensity expression.

FIGS. 14A-14C depict B-cell trajectory inside germinal centers. FIG. 14A shows a spatial projection of selected B-cell clusters from proteins profiles in human tonsil tissues for all imaged follicle regions. FIG. 14B shows pseudotime analysis of B-cells. Left: T-SNE embedding and unsupervised clustering of B-cells. Right: Pseudotime analysis. FIG. 14C shows a projection of single-cell marker intensity on T-SNE map.

FIGS. 15A-15B depict a projection of B-cell trajectory on T-SNE. FIG. 15A shows single-cell T-SNE embedding showing the trajectory of B-cells inside germinal center regions. Arrows showing the trajectory along each cell cluster. FIG. 15B shows a graph-directed plot of B-cell embedding on T-SNE.

FIGS. 16A-16B depict a spatial projection of B-cell trajectory inside germinal centers. Spatial projection of single cell with corresponding pseudotime value from differentiation analysis in tonsil donor A (FIG. 16A) and tonsil donor B (FIG. 16B).

FIGS. 17A-17B depict a spatial projection of B-cell trajectory inside germinal centers. FIG. 17A shows a spatial projection of selected B-cell pseudotime differentiation analysis from germinal center dark zone to germinal center light zone. Left: Arrows showing the trajectory along each cell cluster with corresponding pseudotime value. Right: Spatial projection of single cells with corresponding pseudotime value. FIG. 17B shows a spatial projection of selected B-cell pseudotime differentiation analysis from germinal center dark zone to activated B-cells. Left: Arrows showing the trajectory along each cell cluster with corresponding pseudotime value. Right: Spatial projection of single cells with corresponding pseudotime value.

FIGS. 18A-18C depict VAE embeddings for joint metabolite and protein profiles. FIG. 18A shows a schematic representation of joint embedding reconstruction compared to individual modality reconstruction using VAE for metabolite and protein profiles. FIG. 18B shows a table showing the mean absolute error of reconstruction from joint-VAE and single modality VAE across the imaged datasets. FIG. 18C shows a UMAP showing the VAE joint embedding using common protein markers in lung cancer and tonsil datasets.

FIGS. 19A-19B depict single cell spatial metabolomics (scSpaMet). FIG. 19A shows tissues are stained with isotope-antibody libraries. 3D-SMF (Sci Adv 2021) maps metabolites over 3D at submicron spatial resolution. IMC (Comms Bio 2021) localizes single cells. Registered images reconstruct scSpaMet data. FIG. 19B shows tumor images patterned on a PDMS chip using laser cutting. Bioinspired designs inform tumor chip designs. scSpaMet identifies infiltration around cancer cells confined in the tumor chips.

FIGS. 20A-20D depict spatial metabolic analysis by 3D-SMF. FIG. 20A shows immune cell-specific labeling of a tonsil-sliced sample using an isotope-tagged antibody library. FIG. 20B shows the t-SNE plot exhibited the highest correlated metabolites (blue), anticorrelated metabolites (green), and isotope-tagged antibody labels (black) in the labeled tissue dataset. The calibrated mass spectra consisted of 189 compounds that contained 20 peaks for cell features and 169 peaks for metabolites. FIG. 20C shows pairwise 3D correlations for only the antibody-labeled masses of the isotope antibody-labeled tonsil data using a hierarchically clustered heatmap. FIG. 20D shows two rows of lipid fragment metabolic images were assigned a unique color and then overlaid together with the morphological image. Each immune marker and lipid metabolic image were assigned a unique color and then overlaid.

FIG. 21 depicts spatial metabolomics in unstained lung tumors. Metabolic imaging in squamous lung carcinoma tissue. Yellow channels (PO3) showed cancer cells, surrounded by stroma.

FIG. 22 depicts scSpaMet in lung tumors. Spatial metabolic and cell-protein specific images in lung adenocarcinoma tissue. IMC images indicated immune and cancer cells (blue-red) and nucleus. 3D-SMF images showed lipids and elements. scSpaMet reveals intracellular (colormap: orange) and extracellular (colormap: gray) metabolic distributions.

FIG. 23 depicts lipid metabolism in lung tumors. Fatty acid oxidation pathway in cancer cells and immune cells. 3D-scSpaMet detects metabolites located in FOA pathways.

FIGS. 24A-24C depict a Tumor inspired chip. FIG. 24A shows multiplexed images from lung tumors were used to extract a mask for laser patterning of PDMS films. FIG. 24B shows cellular confined in tumor chips regulate organelles, actins, and cell division, linking to metabolism. FIG. 24C shows single-cell spatial metabolism in a cell located in the chip.

### DETAILED DESCRIPTION

It is appreciated that certain features of the disclosure, which are, for clarity, described in the context of separate aspects, can also be provided in combination with a single aspect. Conversely, various features of the disclosure, which are, for brevity, described in the context of a single aspect, can also be provided separately or in any suitable subcombination. Unless defined otherwise, all technical and scientific terms used herein have the same meaning as commonly understood by one of ordinary skill in the art. Methods and materials similar or equivalent to those described herein can be used in the practice or testing of the present disclosure.

### Definitions

In this specification and in the claims that follow, reference will be made to a number of terms, which shall be defined to have the following meanings:

Throughout the description and claims of this specification, the word “comprise” and other forms of the word, such as “comprising” and “comprises,” means including but not limited to, and are not intended to exclude, for example, other additives, segments, integers, or steps. Furthermore, it is to be understood that the terms comprise, comprising, and comprises as they relate to various aspects, elements, and features of the disclosed invention also include the more limited aspects of “consisting essentially of” and “consisting of.”

As used herein, the singular forms “a,” “an,” and “the” include plural referents unless the context clearly dictates otherwise. Thus, for example, reference to a “polymer” includes aspects having two or more such polymers unless the context clearly indicates otherwise.

Ranges can be expressed herein as from “about” one particular value and/or to “about” another particular value. When such a range is expressed, another aspect includes from the one particular value and/or to the other particular value. Similarly, when values are expressed as approximations, by use of the antecedent “about,” it will be understood that the particular value forms another aspect. It should be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

For the terms “for example” and “such as,” and grammatical equivalences thereof, the phrase “and without limitation” is understood to follow unless explicitly stated otherwise.

### Methods

Here, a single-cell spatial metabolomic (scSpaMet) profiling technology is used and the approach is applied to the native lung cancer biopsies and bioinspired tumor microfluidic chips.

First, tissues are labeled by a 20-plex isotope-barcoded antibody library one-time to enhance the contrast of specific immune and cancer cell types.

Second, 3D-SMFs profile hundreds of lipids and elements in a spatially resolved manner at a submicron resolution over 200-1,000 depth slices. 3D-SMF combined with isotope-tagging identified cell types in metabolic maps, but the cell boundaries were not still definitive due to sensitivity issues of ion conversions.

Finally, the IMC efficiently localizes cytoplasmic and nuclear boundaries to reconstruct metabolic distributions around individual cell types, allowing single-cell mapping in the images.

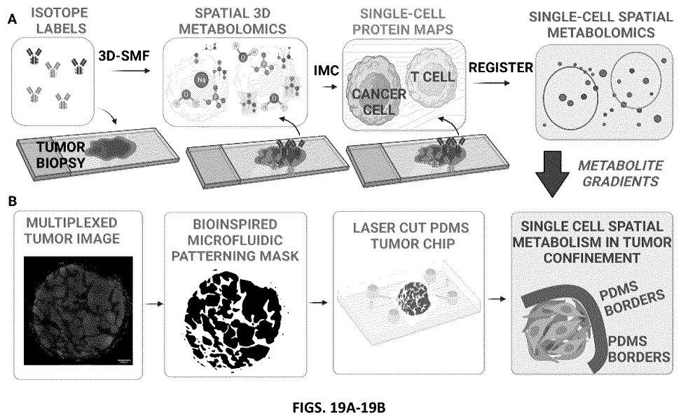

Additionally, a bioinspired tumor chip is used to study the dynamics of immune infiltration as a function of spatially patterned cancer cells within physical confinements, allowing deciphering of the metabolic regulation controlling immune infiltration in a microfluidic chip (FIG. 19B).

In an aspect, provided is a method of detecting analytes in a cell or tissue sample, the method comprising: a) introducing into the cell or tissue sample at least one tagging moiety, wherein the tagging moieties can interact with specific proteins of interest; b) detecting analytes and tagging moieties in the cell or tissue sample; c) spatially detecting proteins in the cell or tissue sample; and d) constructing a map of the analytes in the cell or tissue sample based on the data from steps b) and c).

As used herein, the term “interact” refers to a physical contact between at least two compounds or molecules. In some aspects, the physical contact can be accomplished by one or more of: hydrophobic bonding, van der Waals forces, salt bridges, ionic interactions, electrostatic forces, hydrogen bonding, electron sharing, disulfide bonds, or other suitable intermolecular or intramolecular forces. In some aspects, a given interaction can exist for any length of time depending on the compounds or molecules involved.

As used herein, the term “analyte” refers to any compound, molecule, or other substance of interest to be detected, identified, or characterized. In some aspects, the analyte is a protein, a metabolite, a lipid, a sugar, a carbohydrate, a nucleic acid, an organic molecule, an inorganic molecule, or another suitable compound. In some aspects, the analyte is a protein. In some aspect, the protein is a receptor. As used herein, the term “receptor” refers to a protein which can produce a biological signal in a cell when the protein binds to a specific molecule. In some aspects, the analyte is a metabolite.

As used herein, the term “tagging moiety” refers to any molecule or compound which binds only to a specific motif, functional group, or domain in a compound or molecule of interest. In some aspects, each tagging moiety interacts with a specific protein of interest. In some aspects, the at least one tagging moiety is an isotope-tagged antibody. In some aspects, the method further comprises using two or more tagging moieties simultaneously, wherein each tagging moiety can interact with a different specific protein of interest.

In some aspects, step b) comprises a spatially resolved three-dimensional metabolic profiling framework (3D-SMF). 3D-SMF is an isotope-antibody conjugate labeling for an overnight incubation procedure, followed by secondary ion beam imaging of the biological tissue specimens for reconstructing 3D metabolites and targeted proteins in the same measurement. In some aspects, time-of-flight secondary ion mass spectrometry (TOF-SIMS) is used to perform 3D-SMF. TOF-SIMS utilizes ionization methods exposed to a sample of interest (e.g., hard or soft materials) to produce gas-phase ions, followed by an analysis of mass to charge ratio (m/z) detected by a mass spectral analyzer. The sample of interest is chemically fixed and treated with a sample preparation method (e.g., matrix deposition) and ionized by an electrospray, a laser beam, or an ion beam. The resultant gas ions are then detected by an ion trap mass spectrometer and time-of-flight (TOF) mass spectrometer, revealing the identity of metabolites, lipids, and proteins, along with their spatial coordinates. Secondary ion beam methods incorporate ion bombardments that can vaporize biological specimens, generating secondary ions from the specimens. A TOF detector then defines the metabolites and isotope-tag identities in the same experimental procedure. Ion guns can be selected from Cesium, Bismuth, Argon, Plasma Gun, or other suitable equipment.

As used herein, the term “spatially detecting” refers to confirming the presence of and identifying the spatial location of a molecule, compound, analyte, or complex in a cell or tissue sample. In some aspects, step c) comprises imaging mass cytometry (IMC). Imaging mass cytometry is a specialized used case of TOF detectors in biological specimens ablated by an Ultraviolet (UV) laser, generating also secondary ions for chemical and isotopic analysis in specimens. Alternative laser sources can be used to generate blooms of chemicals integrated with TOF spectral analyzers for such an analysis.

In some aspects, step c) further comprises determining nuclear and cytosolic distributions of analytes. As used herein, the term “nuclear distribution” refers to the spatial and/or temporal arrangement of analytes within the nucleus of a cell. As used herein, the term “cytosolic distribution” refers to the spatial and/or temporal arrangement of analytes within the cytoplasm of a cell.

In some aspects, steps a), b), c) and d) are repeated at multiple spatial locations in the cell or tissue sample. As used herein, “multiple spatial locations” refers to multiple regions of the cell or tissue sample that vary in the x-direction, the y-direction, and/or the z-direction. In some aspects, the multiple spatial locations can cover the entire cell or tissue sample. In some aspects, the multiple spatial locations can cover only a portion of the cell or tissue sample.

In some aspects, steps a), b), c), and d) are repeated at multiple points in time. In some aspects, the multiple points in time can be spread out across minutes, hours, days, weeks, months, or years. In some aspects, the multiple points in time are spread out evenly. In some aspects, the multiple points in time are not spread out evenly.

In some aspects, steps b) and c) are performed sequentially. In some aspects, custom instrument design can allow simultaneous acquisition. The spatial resolution could achieve 100-nm details up to 1-μm. This resolution adjustment is performed based on the signal to noise ratio requirements of detected mass images. sequentially using two instruments or simultaneously using a custom designed instrument. It takes minutes to hours depending on scanning speed and required spatial resolution in the range of 100 nm to 1 μm over the 20×20 μm² to 500×500 μm² areas.

In some aspects, the cell sample comprises cancer cells. Tissues and cells can be obtained from any source. For example, tissues and cells can be obtained from single-cell or multicellular organisms (e.g., a mammal). Tissues and cells obtained from a mammal, e.g., a human, often have varied analyte levels (e.g., gene and/or protein expression) which can result in differences in cell morphology and/or function. The position of a cell or a subset of cells (e.g., neighboring cells and/or non-neighboring cells) within a tissue can affect, e.g., the cell's fate, behavior, morphology, and signaling and cross-talk with other cells in the tissue. In some aspects, the cell sample comprises immune cells. Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells.

In some aspects, the methods described herein can be used in conjunction with any type of cell aggregate or tumor cell. For example, the methods of the invention can identify cancer types or sarcomas. Specific cancers that can be examined and identified using the method of the present invention include, but are not limited to, breast cancer, ovarian cancer, colon cancer, pancreatic cancer, prostate cancer, squamous cell cancer, cervical cancer, lung cancer, small cell lung cancer, kidney cancer, liver cancer, brain tumor, skin cancer, and bladder cancer. In some instances, cancers are derived from xenografts of human cancer cells removed from humans or non-human mammals (e.g., mice).

In some aspects, disclosed are methods that include assessing, scoring, or defining a tumor or cancer in a biological sample. Examples of cancer that can be treated in accordance with the methods described herein include, but are not limited to, B cell lymphomas (e.g., B cell chronic lymphocytic leukemia, B cell non-Hodgkin lymphoma, cutaneous B cell lymphoma, diffuse large B cell lymphoma), basal cell carcinoma, bladder cancer, blastoma, brain metastasis, breast cancer, Burkitt lymphoma, carcinoma (e.g., adenocarcinoma (e.g., of the gastroesophageal junction)), cervical cancer, colon cancer, colorectal cancer (colon cancer and rectal cancer), endometrial carcinoma, esophageal cancer, Ewing sarcoma, follicular lymphoma, gastric cancer, gastroesophageal junction carcinoma, gastrointestinal cancer, glioblastoma (e.g., glioblastoma multiforme, e.g., newly diagnosed or recurrent), glioma, head and neck cancer (e.g., head and neck squamous cell carcinoma), hepatic metastasis, Hodgkin's and non-Hodgkin's lymphoma, kidney cancer (e.g., renal cell carcinoma and Wilms' tumors), laryngeal cancer, leukemia (e.g., chronic myelocytic leukemia, hairy cell leukemia), liver cancer (e.g., hepatic carcinoma and hepatoma), lung cancer (e.g., non-small cell lung cancer and small-cell lung cancer), lymphoblastic lymphoma, lymphoma, mantle cell lymphoma, metastatic brain tumor, metastatic cancer, myeloma (e.g., multiple myeloma), neuroblastoma, ocular melanoma, oropharyngeal cancer, osteosarcoma, ovarian cancer, pancreatic cancer (e.g., pancreatis ductal adenocarcinoma), prostate cancer (e.g., hormone refractory (e.g., castration resistant), metastatic, metastatic hormone refractory (e.g., castration resistant, androgen independent)), renal cell carcinoma (e.g., metastatic), salivary gland carcinoma, sarcoma (e.g., rhabdomyosarcoma), skin cancer (e.g., melanoma (e.g., metastatic melanoma)), soft tissue sarcoma, solid tumor, squamous cell carcinoma, synovia sarcoma, testicular cancer, thyroid cancer, transitional cell cancer (urothelial cell cancer), uveal melanoma (e.g., metastatic), verrucous carcinoma, vulval cancer, and Waldenstrom macroglobulinemia.

A biological sample can have regions that show morphological feature(s) that may indicate the presence of disease or the development of a disease phenotype. For example, morphological features at a specific site within a tumor biopsy sample can indicate the aggressiveness, therapeutic resistance, metastatic potential, migration, stage, diagnosis, and/or prognosis of cancer in a subject. A change in the morphological features at a specific site within a tumor biopsy sample often correlate with a change in the level or expression of an analyte in a cell within the specific site, which can, in turn, be used to provide information regarding the aggressiveness, therapeutic resistance, metastatic potential, migration, stage, diagnosis, and/or prognosis of cancer in a subject. A region or area within a biological sample that is selected for specific analysis (e.g., a region in a biological sample that has morphological features of interest) is often described as “a region of interest.”

In another aspect, provided is a microfluidic chip, comprising microchannels etched into a material, wherein the microchannels comprise a patterned mask of a tumor, wherein the patterned mask of the tumor is obtained by any of the disclosed methods.

As used herein, the term “microfluidic” refers to any system which can manipulate very small volumes of fluids via microchannels, wherein the very small volume of fluid is measured in μL or smaller units of volume measurement. As used herein, the term “microchannel” refers to a channel having a hydraulic diameter of about 1 mm or less. In some aspects, the system is a chip, a reactor, a disc, a slide, a scaffold, or another suitable system for supporting microchannels.

In some aspects, the material is a polymer sheet or hydrogel. In some aspects, the polymer sheet or hydrogel is made from polydimethylsiloxane, polyethylene, polyethylene glycol, polymethyl methacrylate, polyethylene terephthalate, polyester, polystyrene, polycarbonate, polytetrafluoroethylene, or other suitable polymers. In some aspects, the material is glass. In some aspects, the material is quartz. In some aspects, conductive coating such as metal evaporation can be used to enhance the image quality on glass or quartz substrates.

In some aspects, the microfluidic chip is prepared by etching microchannels into the material. In some aspects, the microchannels are etched into the material by laser cutting, physical etching, chemical etching, or other suitable etching methods. In some aspects, the microfluidic chip is prepared by additive manufacturing. In some aspects, the microfluidic chip is prepared by lithography. In some aspects, the microfluidic chip is prepared by hot embossing and imprinting.

In some aspects, the patterning mask recapitulates a spatial organization of a tumor from a patient. As used herein, the term “spatial organization” refers to the distribution of vessels, cells, shape, and/or size of a tissue. In some aspects, the tumor includes cells from lung adenocarcinoma, squamous cell carcinoma, or any other type of cancer cell.

In some aspects, the microfluidic chip further comprises cells. In some aspects, the cells are from the tumor of the patient. In some aspects, the cells are from another patient or cell line.

In some aspects, the cells are cultured in the microchannels. In some aspects, the cells are cultured via adhesion to the walls of the microchannels. In some aspects, the cells are cultured via suspension in a fluid contained within the microchannels.

In another aspect, provided is a method of monitoring an in situ model of a tumor, the method comprising: preparing any of the disclosed microfluidic chips, wherein the microfluidic chip comprises an in situ model of a tumor; and monitoring behavior of the tumor model.

In some aspects, the method further comprises administration of a therapeutic agent, ablation, or method of treatment. In some aspects, the therapeutic agent is an anti-cancer agent, an antibiotic, or a small molecule.

As used herein, the term “ablation” refers to damaging a cell or a portion of tissue physically, thermally, chemically, radiatively, or by any other means suitable to induce cellular damage.

In some aspects, the method of treatment is exposure to radiation.

In some aspects, the microfluidic chip is used for drug screening, evaluation of a method of treatment, evaluation of ablation, development of a customized treatment, disease diagnosis, or other clinical applications.

In some aspects, the tumor model is observed over time. In some aspects, the tumor model is observed over minutes, hours, days, weeks, months, or years. In some aspects, the tumor model is observed at regular intervals. In some aspects, the tumor model is observed at irregular intervals.

In another aspect, provided is a method of detecting cancer in a subject in need thereof, the method comprising: a) introducing into a cell or tissue sample from the subject at least one tagging moiety, wherein the tagging moieties can interact with specific proteins of interest; b) detecting analytes and tagging moieties in the cell or tissue sample; c) spatially detecting proteins in the cell or tissue sample; and d) constructing a map of the analytes in the cell or tissue sample based on the data from steps b) and c).

In some aspects, the analyte is a protein, a metabolite, a lipid, a sugar, a carbohydrate, a nucleic acid, an organic molecule, an inorganic molecule, or another suitable compound. In some aspects, the analyte is a protein. In some aspect, the protein is a receptor. In some aspects, the analyte is a metabolite.

In some aspects, each tagging moiety interacts with a specific protein of interest. In some aspects, the at least one tagging moiety is an isotope-tagged antibody. In some aspects, the method further comprises using two or more tagging moieties simultaneously, wherein each tagging moiety can interact with a different specific protein of interest.

In some aspects, step b) comprises a spatially resolved three-dimensional metabolic profiling framework (3D-SMF). In some aspects, time-of-flight secondary ion mass spectrometry (TOF-SIMS) is used to perform 3D-SMF.

In some aspects, step c) comprises imaging mass cytometry (IMC).

In some aspects, step c) further comprises determining nuclear and cytosolic distributions of analytes.

In another aspect, provided is a method of determining response of a subject to a treatment protocol, the method comprising monitoring a cell or tissue sample from the subject across multiple points in time, wherein monitoring the cell or tissue sample at each point in time comprises: a) introducing into a cell or tissue sample from the subject at least one tagging moiety, wherein the tagging moieties can interact with specific proteins of interest; b) detecting analytes and tagging moieties in the cell or tissue sample; c) spatially detecting proteins in the cell or tissue sample; and d) constructing a map of the analytes in the cell or tissue sample based on the data from steps b) and c).

In some aspects, the analyte is a protein, a metabolite, a lipid, a sugar, a carbohydrate, a nucleic acid, an organic molecule, an inorganic molecule, or another suitable compound. In some aspects, the analyte is a protein. In some aspect, the protein is a receptor. In some aspects, the analyte is a metabolite.

In some aspects, each tagging moiety interacts with a specific protein of interest. In some aspects, the at least one tagging moiety is an isotope-tagged antibody. In some aspects, the method further comprises using two or more tagging moieties simultaneously, wherein each tagging moiety can interact with a different specific protein of interest.

In some aspects, step b) comprises a spatially resolved three-dimensional metabolic profiling framework (3D-SMF). In some aspects, time-of-flight secondary ion mass spectrometry (TOF-SIMS) is used to perform 3D-SMF.

In some aspects, step c) comprises imaging mass cytometry (IMC).

In some aspects, step c) further comprises determining nuclear and cytosolic distributions of analytes.

In some aspects, steps a), b), c) and d) are repeated at multiple spatial locations in the cell or tissue sample. In some aspects, the multiple spatial locations can cover the entire cell or tissue sample. In some aspects, the multiple spatial locations can cover only a portion of the cell or tissue sample.

In some aspects, steps a), b), c), and d) are repeated at multiple points in time. In some aspects, the multiple points in time can be spread out across minutes, hours, days, weeks, months, or years. In some aspects, the multiple points in time are spread out evenly. In some aspects, the multiple points in time are not spread out evenly.

### EXAMPLES

### Example 1—Methods

Tissue preparation and isotope-conjugates antibody labeling: Patients' samples for lung tumor were obtained from a tumor microarray (TMA) purchased from a third-party vendor (Biomax, US) with the tissue ID: BS04081a. This TMA included a total of 63 tissue cores of formalin-fixed paraffin-embedded (FFPE) non-small cell lung adenocarcinoma and adjacent normal lung tissue samples obtained from 21 patients. 21 regions of interest from 7 cores were imaged. Each tissue core had a diameter of 1 mm and a thickness of 5-μm which is within the tissue thickness recommended for IMC (≤7-μm). The tissue labeling protocol was followed as previously reported in the protocol³⁵ including antigen retrieval, protein blocking, metal-tagged antibody labeling, and nucleus counterstains. After the staining process is complete, the stained tissues were stored at 4° C. until imaging time. The human tonsil tissue sections were from TissueArray.com under the IDs HuFPT161. Tonsil sample 1 had tissue ID SU1 and tonsil sample 2 had tissue ID SM2. Tonsil sample 1 had 5 imaging regions of interest and tonsil sample 2 had 6 imaging regions of interest.

IMC imaging: To set up the Hyperion imaging system, regions of interest (ROIs) of 1500-μm×1500-μm were chosen within each tissue core to cover most of the tissue. To choose the most optimum laser ablation power, several testing points were chosen from the tissue cores that represent the tissue heterogeneity. The acquired data is automatically saved in .mcd format that can be viewed using Fluidigm's MCD Viewer software (v 1.0.560.2).

IMC image processing: Each ROI image was extracted using MCD Viewer (v 1.0.560.2) with minimum threshold intensity of 0 and maximum threshold intensity of 50. Each image intensity range was then scaled to 0.1 and 99.9th intensity percentile for processing. Noise removal using a KNN filter⁷⁷ is applied to reduce noise in the dataset.

TOF-SIMS imaging: The TOF-SIMS (IONTOF 5 GmbH, Munster, Germany) instrument uses a bismuth liquid metal ion gun as the primary ion source to generate secondary ions from the sample surface, followed by identification of m/z of secondary ions by a TOF analyzer. The bismuth source can be used in different modes: Singly charged 25-kV monoatomic (Bi⁺), singly charged 25-kV three-atom cluster (Bi₃⁺), or doubly charged 50-kV cluster (Bi₃⁺⁺) mode. Distinct modes exhibit extra cluster mass and energy that can increase the yield (i.e., the number of secondary ions per primary ion) of heavier molecular weight secondary ion species. The Bi₃⁺⁺ mode was chosen based on single-cell features. Regions of 300×300 to 400×400 μm² area were raster scanned at 512×512 pixels. Pixel densities were chosen empirically. The bright field images were used to find imaging regions identified in H & E sequential tissues. In this device, the secondary ions were collected and then accelerated across a voltage gap and directed into an ultrahigh-vacuum flight tube toward the detector (a combination channel plate and photomultiplier tube). The flight time of secondary ions is proportional to the ion m/z; thus, lighter ions (lower m/z) arrive at the detector more quickly, and the heavier the ion, the longer the TOF. For depth profiling, another cesium ion gun (Cs⁺ ions, 2-kV energy, and microampere current) was used to iteratively sputter away very thin layers, followed by bismuth (Bi) bombardment to generate secondary ions from the tissue sample before and after Cs sputtering cycles. Depth profiling allowed the detection of more molecules in imaging regions. Depth profiling in the TOF-SIMS was performed around 30-40 slide for 10 s ablation at 2-kV per slice for an estimate ablation of 1 micron per hour. In the 3D-SMF platform, negative and positive modes were experimentally tested. As the metabolomic profiling measurements in unlabeled samples provided more noteworthy single-cell spatial distributions in the negative mode, the rest of the experiments were performed to detect the negatively charged compounds as the selection of polarity.

SIMS data preprocessing: The IONTOF SurfaceLab software (version 6) was used to perform basic image processing operations on the acquired spatial mass spectra. The spatial distribution for each selected peak was exported in files containing the coordinates and pixel intensity values. Around 200 peaks were selected using Surface Lab. The data were then exported in American Standard Code for Information Interchange (ASCII) format into a text file. For putative annotation of the metabolite channels, TOF-SIMS metabolite imaging literature was used (TABLE 1, TABLE 2). For SIMS data, the mean ion count normalization was applied for intra-sample normalization⁷⁸. Each pixel in the imaging data is normalized by their mean intensity over all extracted ion channels. If the pixel i in the imaging data is represented by a vector Xᵢ=(x_{i,1}, . . . , x_{i,p})∈R^{p}, the normalization is defined as

$${Y}_{i} = \frac{X i}{{Σ}_{j = i}^{p} {x}_{ij}} P .$$

TABLE 1
Putative annotation of TOF-SIMS channel sort by m/z.

| m/z | Ion | Molecules | Type |
|---|---|---|---|
| 25.01 | | L-Frag | Metabolite |
| 26 | | L-Frag | Metabolite |
| 30 | | L-Frag | Metabolite |
| 39 | | K | |
| 41 | | L-Frag | Metabolite |
| 42 | CNO− | L-Frag | Metabolite |
| 44.998 | | L-Frag | Metabolite |
| 53 | | L-Frag | Metabolite |
| 54 | | L-Frag | Metabolite |
| 55 | | L-Frag | Metabolite |
| 56 | | L-Frag | Metabolite |
| 57 | | L-Frag | Metabolite |
| 58.005 | | L-Frag | Metabolite |
| 59 | C3H9N | Glycerophosphocholines | Metabolite |
| 64 | | L-Frag | Metabolite |
| 65 | | L-Frag | Metabolite |
| 66 | | L-Frag | Metabolite |
| 67 | | L-Frag | Metabolite |
| 68 | | L-Frag | Metabolite |
| 69 | | L-Frag | Metabolite |
| 70 | | L-Frag | Metabolite |
| 72 | | L-Frag | Metabolite |
| 74 | | Glycine | Amino Acid |
| 78.96 | PO3− | Phosphate | Metabolite |
| 80 | | L-Frag | Metabolite |
| 81 | | L-Frag | Metabolite |
| 82 | | L-Frag | Metabolite |
| 83 | | L-Frag | Metabolite |
| 84 | | L-Frag | Metabolite |
| 85.029 | | L-Frag | Metabolite |
| 86 | C5H12N | Glycerophosphocholines | Metabolite |
| 87 | C3H3O3− | Pyruvic acid | Metabolite |
| 88 | | Alanine | Amino Acid |
| 89.02 | C3H5O3− | Lactic acid | Metabolite |
| 91 | | L-Frag | Metabolite |
| 93.03 | | L-Frag | Metabolite |
| 95 | C7H11− | Cholesterol fragment | Cholesterol fragment |
| 96 | | L-Frag | Metabolite |
| 98 | | L-Frag | Metabolite |
| 99.045 | | L-Frag | Metabolite |
| 100 | | L-Frag | Metabolite |
| 102 | C5H12NO | Ceramide phosphocholines | Metabolite |
| 103 | | L-Frag | Metabolite |
| 104.03 | C3H6NO3− | Serine | Amino Acid |
| 109 | C8H13 | Cholesterol fragment | Cholesterol fragment |
| 110.03 | C4H4N3O− | Cytosine | Metabolite |
| 111.02 | C4H3N2O2− | Uracil | Metabolite |
| 113.06 | | L-Frag | Metabolite |
| 114.05 | C5H8NO2− | Proline | Amino Acid |
| 116.07 | C5H10NO2− | Valine | Amino Acid |
| 118 | | Threonine | Amino Acid |
| 120 | | Cysteine | Amino Acid |
| 124.05 | C5H6N3O− | Methylcytosine | Metabolite |
| 125.03 | C5H5N2O2− | Thymine | Metabolite |
| 126 | C2H9NPO3 | Glycerophosphoethanolamines | Metabolite |
| 127.07 | | L-Frag | Metabolite |
| 130.08 | C6H12NO2− | Isoleucine/leucine | Amino Acid |
| 131.04 | C4H7N2O3 | Asparagine | Amino Acid |
| 132 | | Aspartic acid/Ornithine | Amino Acid |
| 134.04 | C5H4N5 | Adenine | Metabolite |
| 135.03 | C5H3N4O− | Hypoxanthine | Metabolite |
| 141.092 | | L-Frag | Metabolite |
| 142 | C2H9NPO4 | Glycerophosphoethanolamines | Metabolite |
| 145.06 | C5H9N2O3− | Glutamine | Amino Acid |
| 146.04 | C5H8NO4− | Glutamic acid | Amino Acid |
| 147 | C11H15 | Cholesterol fragment | Cholesterol fragment |
| 148 | | Methionine | Amino Acid |
| 150.04 | C5H4N5O− | Guanine | Metabolite |
| 152.07 | C8H10NO2− | Dopamine | Metabolite |
| 152.99 | C3H6PO5− | Glycerol-3-phosphate | Metabolite |
| 154.06 | C6H8N3O2− | Histidine | Amino Acid |
| 155.1 | | L-Frag | Metabolite |
| 156.03 | C6H6NO4− | Aspartic acid | Metabolite |
| 161 | C12H17 | Cholesterol fragment | Cholesterol fragment |
| 163 | | L-Frag | Metabolite |
| 164.07 | C9H10NO2− | Phenylalanine | Amino Acid |
| 165 | C10H13O2 | Hydroquinone | Metabolite |
| 166 | C5H13NPO3 | Glycerophosphocholines | Metabolite |
| 167.1 | | L-Frag | Metabolite |
| 168.04 | C4H11NO4P− | Phosphocholine-CH3 | Metabolite |
| 169.123 | | L-Frag | Metabolite |
| 171.1 | C10H20O2− | FA(10:0) | Faty acid |
| 173 | | Arginine | Amino Acid |
| 180.06 | C9H10NO3− | Tyrosine | Amino Acid |
| 181.123 | | L-Frag | Metabolite |
| 183.138 | | L-Frag | Metabolite |
| 184 | C5H15NPO4 | Glycerophosphocholines | Metabolite |
| 195.138 | | L-Frag | Metabolite |
| 197.154 | | L-Frag | Metabolite |
| 199.17 | C12H23O2− | FA(12:0) | Faty acid |
| 203.08 | C11H11N2O2 | Tryptophan | Amino Acid |
| 205 | C11H11N2O3 | Hydroquinone | Metabolite |
| 206 | C5H14PO4N | Glycerophosphocholines | Metabolite |
| 209.154 | | L-Frag | Metabolite |
| 219 | | Glucose | |
| 221.154 | | L-Frag | Metabolite |
| 224 | C8H19NPO4 | Glycerophosphocholines | Metabolite |
| 227.18 | C14H27O2− | FA(14:0) | Faty acid |
| 237.185 | | L-Frag | Metabolite |
| 246 | C8H19NPO4 | Glycerophosphocholines | Metabolite |
| 251.2 | C16H27O2− | FA(16:2) | Faty acid |
| 253.2 | C16H29O2− | FA(16:1) | Faty acid |
| 255.23 | C16H31O2− | FA(16:0) | Faty acid |
| 267.07 | C10H11N4O5− | Inosine | Metabolite |
| 275.2 | C18H27O2− | FA(18:4) | Faty acid |
| 277.21 | C18H29O2− | FA(18:3) | Faty acid |
| 279.22 | C18H31O2− | FA(18:2) | Faty acid |
| 281.25 | C18H33O2− | FA(18:1) | Faty acid |
| 283.35 | C18H35O2− | FA(18:0) | Faty acid |
| 289.26 | C20H40NO4S− | C18 taurine | Metabolite |
| 303.23 | C20H31O2− | FA(20:4) | Faty acid |
| 305.25 | C20H33O2− | FA(20:3) | Faty acid |
| 307.26 | C20H35O2− | FA(20:2) | Faty acid |
| 309.28 | C20H37O2− | FA(20:1) | Faty acid |
| 311.29 | C20H39O2− | FA(20:0) | Faty acid |
| 328.04 | C10H11N5O6P− | cAMP | Metabolite |
| 346.05 | C10H13N5O7P− | AMP | Metabolite |
| 362.05 | C10H13N5O8P− | GMP | Metabolite |
| 369.3 | C27H45 | Cholesterol fragment | Cholesterol fragment |
| 385.3 | C27H46O | Cholesterol fragment | Cholesterol fragment |
| 388.25 | C20H38NO4S− | C18:1 taurine | Metabolite |

TABLE 2
Putative annotation of TOF-SIMS channel sort by literature reference.

| m/z | Ion | Molecules | Ref. |
|---|---|---|---|
| 42 | CNO− | Small molecular fragment | 1, 2 |
| 78.96 | PO3− | Phosphate | 1, 2 |
| 87 | C3H3O3− | Pyruvic acid | 1, 2 |
| 89.02 | C3H5O3− | Lactic acid | 1, 2 |
| 104.03 | C3H6NO3− | Serine | 1, 2 |
| 110.03 | C4H4N3O− | Cytosine | 1, 2 |
| 111.02 | C4H3N2O2− | Uracil | 1, 2 |
| 114.05 | C5H8NO2− | Proline | 1, 2 |
| 116.07 | C5H10NO2− | Valine | 1, 2 |
| 124.05 | C5H6N3O− | Methylcytosine | 1, 2 |
| 125.03 | C5H5N2O2− | Thymine | 1, 2 |
| 130.08 | C6H12NO2− | Isoleucine/leucine | 1, 2 |
| 131.04 | C4H7N2O3 | Asparagine | 1, 2 |
| 134.04 | C5H4N5 | Adenine | 1, 2 |
| 135.03 | C5H3N4O− | Hypoxanthine | 1, 2 |
| 145.06 | C5H9N2O3− | Glutamine | 1, 2 |
| 146.04 | C5H8NO4− | Glutamic acid | 1, 2 |
| 150.04 | C5H4N5O− | Guanine | 1, 2 |
| 152.07 | C8H10NO2− | Dopamine | 1, 2 |
| 154.06 | C6H8N3O2− | Histidine | 1, 2 |
| 156.03 | C6H6NO4− | Aspartic acid | 1, 2 |
| 164.07 | C9H10NO2− | Phenylalanine | 1, 2 |
| 168.04 | C4H11NO4P− | Phosphocholine-CH3 | 1, 2 |
| 171.01 | | Capric acid | 1, 2 |
| 180.06 | C9H10NO3− | Tyrosine | 1, 2 |
| 203.08 | C11H11N2O2 | Tryptophan | 1, 2 |
| 267.07 | C10H11N4O5− | Inosine | 1, 2 |
| 328.04 | C10H11N5O6P− | cAMP | 1, 2 |
| 346.05 | C10H13N5O7P− | AMP | 1, 2 |
| 362.05 | C10H13N5O8P− | GMP | 1, 2 |
| 388.25 | C20H38NO4S− | C18:1 taurine | 1, 2 |
| 289.26 | C20H40NO4S− | C18 taurine | 1, 2 |
| 199.17 | C12H23O2− | FA(12:0) | 1, 2 |
| 227.18 | C14H27O2− | FA(14:0) | 1, 2 |
| 251.2 | C16H27O2− | FA(16:2) | 1, 2 |
| 253.2 | C16H29O2− | FA(16:1) | 1, 2 |
| 255.23 | C16H31O2− | FA(16:0) | 1, 2 |
| 275.2 | C18H27O2− | FA(18:4) | 1, 2 |
| 277.21 | C18H29O2− | FA(18:3) | 1, 2 |
| 279.22 | C18H31O2− | FA(18:2) | 1, 2 |
| 281.25 | C18H33O2− | FA(18:1) | 1, 2 |
| 283.35 | C18H35O2− | FA(18:0) | 1, 2 |
| 153 | C3H6PO5− | | 1, 2 |
| 255.2 | C16H31O2− | | 1, 2 |
| 281.2 | C18H33O2− | | 1, 2 |
| 95.08 | | Cholesterol | 3 |
| 109.1 | | Cholesterol | 3 |
| 147.11 | | Cholesterol | 3 |
| 161.13 | | Cholesterol | 3 |
| 369.3 | | Cholesterol | 3 |
| 171.01 | | Capric acid | 4 |
| 198.96 | | Lauric Acid | 4 |
| 253.25 | | Palmitoleic acid | 4 |
| 255.26 | | Palmitic acid | 4 |
| 279.26 | | Linoleic acid | 4 |
| 281.2 | | Oleic acid | 4 |
| 255.2 | | FA(16:0) | 5 |
| 283.2 | | FA(18:0) | 5 |
| 281.2 | | FA(18:1) | 5 |
| 279.2 | | FA(18:2) | 5 |
| 59 | C3H9N | Glycerophosphocholines | 5 |
| 86 | C5H12N | Glycerophosphocholines | 5 |
| 104 | C5H14NO | Glycerophosphocholines | 5 |
| 166 | C5H13NPO3 | Glycerophosphocholines | 5 |
| 184 | C5H15NPO4 | Glycerophosphocholines | 5 |
| 206 | C5H14PO4Na | Glycerophosphocholines | 5 |
| 224 | C8H19NPO4 | Glycerophosphocholines | 5 |
| 246 | C8H18NPO4Na | Glycerophosphocholines | 5 |
| 126 | | Glycerophosphoethanolamines | 5 |
| 142 | | Glycerophosphoethanolamines | 5 |
| 86 | | Sphingolipids | 5 |
| 102 | | Sphingolipids | 5 |
| 104 | | Sphingolipids | 5 |
| 184 | | Sphingolipids | 5 |
| 206 | | Sphingolipids | 5 |
| 126 | | Sphingolipids | 5 |
| 142 | | Sphingolipids | 5 |
| 165 | | Sphingolipids | 5 |
| 205 | | Sphingolipids | 5 |
| 30 | | lipid fragment (CH4N+) | 6 |
| 34 | | lipid fragment (C2D5+) | 6 |
| 44 | | lipid fragment (C2H6N+) | 6 |
| 55 | L&C | lipid fragment (C4H7+), arginine, lysine, cysteine, valine, leucine, histidine | 6 |
| 56 | | lipid fragment (C3H6N+), lysine, methionine, glutamine, asparagine, | 6 |
| | | threonine, isoleucine | |
| 57 | L&C | lipid fragment (C4H9+), valine, serine, alanine, arginine, cysteine, aspartic | 6 |
| | | acid, leucine, threonine, glycine | |
| 58 | DLPC | lipid fragment (C3H8N+), isoleucine | 6 |
| 59 | DLPC | lipid fragment (13C12C2H8N+), arginine, valine | 6 |
| 60 | | lipid fragment (C3H10N+), serine | 6 |
| 66 | | lipid fragment (C5H7+) | 6 |
| 67 | | lipid fragment (C5H7+) | 6 |
| 68 | | lipid fragment (C4H6N+), proline | 6 |
| 69 | | lipid fragment (C5H9+), isoleucine, histidine, lysine | 6 |
| 70 | | lipid fragment (C4H8N+), proline, leucine, glutamic acid, asparagine, | 6 |
| | | arginine | |
| 71 | | lipid fragment (C5H11+) | 6 |
| 72 | | lipid fragment (C4H10N+), valine | 6 |
| 74 | | lipid fragment (C4H12N+) threonine | 6 |
| 81 | | lipid fragment (C6H9+), alanine | 6 |
| 82 | | lipid fragment (C5H8N+), histidine | 6 |
| 83 | | lipid fragment (C6H11+) | 6 |
| 84 | | lipid fragment (C5H10N+), lysine, glutamine, glutamic acid | 6 |
| 85 | | lipid fragment (C6H13+) | 6 |
| 86 | DLPC | lipid fragment (C5H12N+), isoleucine, leucine | 6 |
| 88 | | lipid fragment (C5H14N+), aspartic acid | 6 |
| 91 | L&C | lipid fragment (C7H7+), serine, phenylalanine, methionine | 6 |
| 93 | | lipid fragment (C7H9+) | 6 |
| 95 | L&C | lipid fragment (C7H11+), histidine | 6 |
| 97 | | lipid fragment (C7H13+) | 6 |
| 98 | | lipid fragment (C5H8NO+), glycine | 6 |
| 100 | | lipid fragment (C5H10NO+) | 6 |
| 102 | | lipid fragment (C5H12NO+), glutamic acid | 6 |
| 104 | | lipid fragment (C5H14NO+), lysine | 6 |
| 105 | L&C | lipid fragment (C5H1415NO+) | 6 |
| 111 | | lipid fragment (C8H15+) | 6 |
| 146 | | lipid fragment (C5H9NPO2+) | 6 |
| 148 | | lipid fragment (C5H11NPO2+), glutamic acid | 6 |
| 166 | | lipid fragment (C5H13NPO3+), methionine, phenylalanine | 6 |
| 167 | | | |
| 168 | | lipid fragment (C5H15NPO3+) | 6 |
| 182 | | lipid fragment (C5H13NPO4+), tyrosine | 6 |
| 184 | DLPC | lipid fragment (C5H15NPO4+) | 6 |
| 185 | | | 6 |
| 190 | | lipid fragment (C7H13NPO3+) | 6 |
| 194 | | lipid fragment (C7H17NPO3+) | 6 |
| 196 | | lipid fragment (C6H15NPO4+) | 6 |
| 198 | | lipid fragment (C6H17NPO4+) | 6 |
| 206 | | lipid fragment (C5H14NPO4Na+) | 6 |
| 210 | | lipid fragment (C7H17NPO4+) | 6 |
| 212 | | lipid fragment (C7H19NPO4+) | 6 |
| 224 | | lipid fragment (C7H15NPO5+) | 6 |
| 226 | | lipid fragment (C7H17NPO5+) | 6 |
| 238 | | lipid fragment (C8H17NPO5+) | 6 |
| 240 | | lipid fragment (C8H19NPO5+) | 6 |
| 246 | | lipid fragment (C8H18NPO4Na+ or C8H18NPO5Li+) | 6 |
| 252 | | lipid fragment (C8H15NPO6+) | 6 |
| 254 | | lipid fragment (C8H17NPO6+) | 6 |
| 256 | | lipid fragment (C8H19NPO6+) | 6 |
| 282 | | lipid fragment (C9H17NPO7+) | 6 |
| 71 | | Glucose fragment | 7 |
| 87 | | Glucose fragment | 7 |
| 99 | | Glucose fragment | 7 |
| 119 | | Glucose fragment | 7 |
| 141 | | Glucose fragment | 7 |
| 159 | | Glucose fragment | 7 |
| 177 | | Glucose fragment | 7 |
| 179 | | Glucose fragment | 7 |
| 359 | | Glucose fragment | 7 |

Pixel clustering: Pixels in each imaged region of interest are concatenated and were down-sampled by 20 folds to extract a subsampling of the whole dataset. The selected pixels were normalized to a mean of 0 and a standard deviation of 1. Then, a Leiden clustering algorithm was applied to the down-sampled dataset to assign a cluster label to each pixel. Finally, pixels of the entire dataset were assigned to the cluster of most likely neighbors among the 30 nearest neighbors in pixel feature space in the down-sampled dataset.

Image registration: A two-step image registration was performed to match and align cross-modality images (SIMS, IMC, H & E). First using a Fast Fourier transform (FFT) algorithm the cross-correlation between two images is calculated to get translational offsets. The position of the maximal correlation coefficient was identified and used to match images. After finding a translational offset and matching region, the rotation offset between the two aligned and cropped images is calculated using FFT on the polar space transformed of the images. Finally, the affine transformation of the two images is found by applying a Difference of Gaussian (DoG) filter local maxima above a selected threshold were selected as features and matched with the RANSAC algorithm⁷⁹. To match the pixel density of the two modalities, the higher-density SIMS modality was down-sampled using bi-quadratic interpolation without anti-aliasing.

Single-cell segmentation: In cancer tissues, single-cell nuclei regions were also segmented using the deep learning model Cellpose⁸⁰ by using Histone H3 marker in lung cancer samples, combining DNA1, DNA2, Ki67, and PD1 markers in tonsil samples and combining DNA1, DNA2 markers in endometrium samples from IMC modality. The cytosol region was calculated by expanding the nuclei-segmented region by 2 pixels. In tonsil tissues, the best available nuclei marker was used (Histone H3 in tonsil data), and multiple protein markers were combined using maximum projection to get the whole cell area (in tonsil data: CD38, Vimentin, CD21, BCL6, ICOS1, CD11b, CD86, CXCR4, CD11c, FoxP3, CD4, CD138, CXCR5, CD20, CD8, C-Myc, PD1, CD83, Ki67, COL1, CD3, CD27, EZH2). To get the cytosolic region of each cell, the nuclei image was subtracted from the combined protein marker image. Finally, deepcell⁸¹ Mesmer model was used for single-cell nuclei and cytosol segmentation. This 2-step segmentation pipeline better captures the overall cytosolic region of single cells by incorporating the majority of available multiplex imaging panels. The combined multiplex images were used for single-cell cytosol and nuclei segmentation (a-c). It was compared to a segmentation using a registered PO3-channel image from 3D-SMF imaging which provided nuclei signals in cells. It was shown that the average captured nuclei area using the IMC modality yields higher cell areas compared to 3D-SIMS and 2-fold higher in IMC cytosol segmentation compared to SIMS nuclei.

Clustering algorithm: Single-cell unsupervised clustering was performed using the Leiden algorithm³⁷ a graph-based community detection algorithm. From each segmented cell region, the mean intensity of each marker expression was calculated. The resulting feature matrix consisted of n rows of the total number of cells (n=19507 for lung, n=31156 for tonsil, n=8215 for endometrium) and p columns of marker expression. Each column of the feature matrix was z-score normalized and batch correction between samples was performed using Scanorama pipeline²⁰. The neighborhood graph in the embedding space was constructed and used for unsupervised community detection. Each cell was associated with a cell phenotype cluster and attributed a cluster color showing the cell-level clustering of each ROI.

Spatial neighboring map: From single-cell segmentation and clustering, cell centroids, and corresponding clusters were extracted. Spatial Neighboring maps were created by connecting centroids within 20-μm of each other in lung cancer and touching cell masks in tonsil samples, thus generating a spatial proximity network for each ROI with corresponding cell phenotype. The average minimum and maximum axis length of single cells were used to infer a mean diameter of 10 μm and a distance of 20 μm was chosen based on 1 cell distance. Cell contacts are extracted by binary dilatation of single-cell masks and cells that overlap was considered to have contact³¹. Cell mask contact was chosen for spatial neighboring maps generation in tonsil samples because of the higher density of cells.

Local cell competition: Cell local metabolite competition is defined by cell spatial neighboring map. For each cell, the metabolite competition ratio was defined as the metabolite expression of the cell divided by the average metabolite expression of its neighboring cells. This gives a metabolite competition ratio equal to 1 when the metabolite of a cell is equal to the average of its neighboring cells, a competition ratio less than 1 when the metabolite of a cell is less than the average of its neighboring cells, and a competition ratio greater than 1 when the metabolite of a cell is greater than the average of its neighboring cells. Let mᵢ be the metabolite expression level of cell i and m_{Ni} the average metabolite expression level of cell i neighbors, the cell metabolite competition ratio is defined as:

$$\frac{{m}_{i}}{{m}_{Ni}} .$$

Variational autoencoder embedding: For protein and metabolites data integration at the single cell level, a Variational autoencoder (VAE) was used for embedding extraction⁴¹. The VAE has input x for single cell, f_{E}, and f_{D} represent the transformation by encoder and decoder layers. In addition to the standard autoencoder, two transformations f_{μ} and f_{σ} are added to the output e of the encoder to generate the parameters μ and σ (μ, σ∈R^{m}). The compressed data z is now sampled from the normal distribution with mean μ and standard deviation σ. In contrast to the standard autoencoder, VAE uses z as the input of the decoder instead of e. By adding randomness in generating z, VAE prevents overfitting by avoiding mapping the original data to the compressed space without learning a generalized representation of data. Formally given an input dataset x the characteristics of z can be inferred by learning the posterior distribution p(z|x) with likelihood distribution p(x|z). The likelihood function p(x|z) is learned with the decoder. The posterior distribution p(z|x) is learned through variational inference by the encoder q. The distribution of z is learned with the re-parameterization trick to ensure model gradient backpropagation by considering the latent space as multivariate gaussian distributions. The VAE implementation maximizes the evidence of lower bound (ELBO) during training:

L(X)=E_{(q(z|x,ϕ))}[“log”p(x|z,θ)]−KL(q(z|x,ϕ)∥p(z))

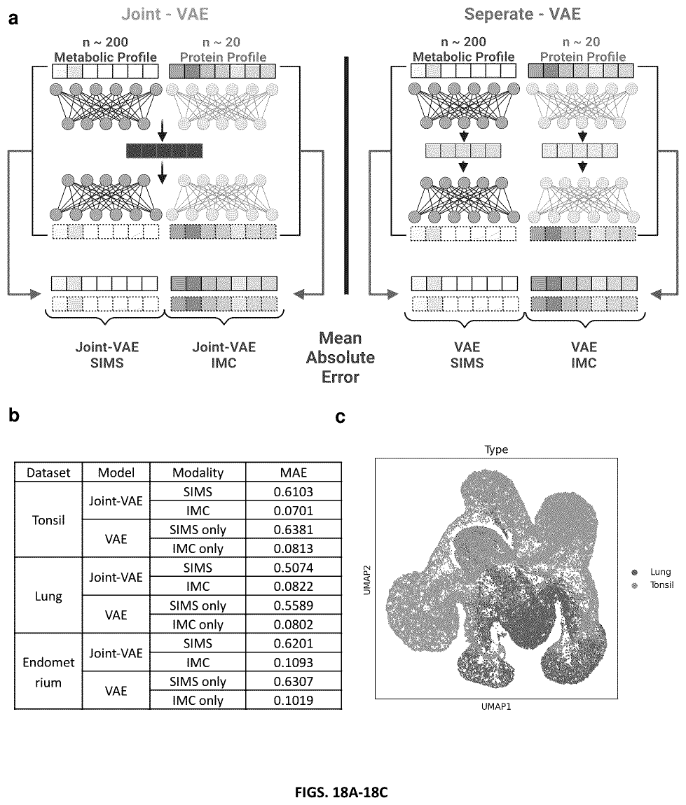

The variational autoencoder consists of two encoder-decoder networks for proteomic (AEₚ) and metabolomic (AEₘ) with a different number of layers and layer embedding sizes. The AEₚ consists of a 2-layer encoder of embedding sizes 16 and 8 respectively and a decoder of embedding sizes 16, and 21 (proteomics dimension size). The AEₘ consists of a 3-layer encoder of embedding sizes 128, 64, and 32, respectively, and a decoder of embedding sizes 64, 128, and 200 (metabolomics dimension size). The joint embedding h is obtained by concatenating the output of the encoder from AEₚ and AEₘ feed-forward to a dense layer of embedding size 16. Then h is used to derive the normal distribution with mean μ and standard deviation σ. During training, to learn a joint embedding from metabolic and protein profiles, two separate encoder-decoder networks were trained: one for metabolic and one for protein profile reconstruction. The two networks share the same embedding space by concatenating the output of the two network encoders and sampling from the same distribution. The joint embedding VAE reconstruction mean absolute error was compared with single modality VAE and showed similar ability for single modality reconstruction with both models (FIGS. 18A-18B). Moreover, when combining lung cancer and tonsil dataset with common metabolite and protein markers, VAE successfully separated the two tissue types (FIG. 18C).

Pseudotime analysis: Single cell pseudotime analysis was performed using protein markers for cell type definition. Two methods, Palentir⁷¹, and Diffusion pseudotime²⁹ were compared to capture the cell trajectory of B cells inside the germinal center from protein data. Pseudotime based on single-cell protein marker phenotypes was used to correlate single-cell metabolite across B-cell germinal center trajectories.

### Example 2—Single-Cell Spatial Metabolomics with Cell-Type Specific Protein Profiling for Tissue Systems Biology

With the advent of the current immunotherapy approaches, it is becoming critical to develop a comprehensive understanding of immune metabolism. The multi-omics scSpaMet approach has the great potential to link the multi-layer information of the proteomics data with the metabolism data on the same biological tissue. The scSpaMet starts with staining the tissues with the metal-isotope conjugated antibodies, performing the metabolic profiling using the Time-of-Flight Secondary Ion Mass Spectrometry (ToF-SIMS) imaging, and finally performing the proteomic profiling using Imaging Mass Cytometry (IMC). 3D-SMF¹⁸ has been developed to profile hundreds of metabolic fragments' mass spectrum peaks in tonsils using ToF-SIMS at the tissue level and the protein expression profile at the single cell level of immune cells in tonsil and lung tissues using IMC^{35,36}. Every multiplexed imaging region in the SIMS data has a resolution of better than 1 μm per pixel for over 200 m/z peaks. Further, IMC provides targeted multiplex protein imaging data for deciphering distinct cell types (for instance, cancer/epithelial, stroma, immune) at 1 μm per pixel resolution for up to 40 markers. Compared to existing metabolomic profiling methods, scSpaMet allows multiplex cell types to metabolic profile correlation at the single-cell level. Compared to 3D-SMF, the scSpaMet imaging pipeline incorporated in situ sequential detection of metabolomic and proteomic within the same tissue (TABLE 3), providing correlative proteomics/metabolomics analysis at the single-cell level by cross-modality spatial registration. Accurate single-cell segmentation from the scSpaMet pipeline allowed single-cell level joint metabolite and protein downstream analysis, whereas 3D-SMF only allowed metabolite channel-level correlation, channel embedding and pixel clustering from tissue regions.

TABLE 3
Comparison of emerging single-cell spatial metabolomics technologies.

| | | | | | Cell | |
|---|---|---|---|---|---|---|
| | | Mass | | Cell type | target | Cell size |
| Method | Capability | imaging | Resolution | specificity | region | compatibility |
| scSpaMet | Integrates | TOF- | 500-nm | 25 Cell | Cytosol | Small cell size |
| | metabolite and | SIMS/IMC | resolution | types | and | |
| | specific | | | | nucleus | |
| | protein | | | | | |
| | labeling at | | | | | |
| | single-cell | | | | | |
| | level | | | | | |
| 3D-SMF | Detect tissue | TOF-SIMS | 500-nm | 20 protein | Tissue | None |
| | region | | resolution | markers | region | |
| | metabolites | | | | | |
| SpaceM | Integrates | MALDI | 10-μm | 2 Cell | Whole | Large cell size |
| | MALDI with | | resolution | types | cell | |
| | confocal | | | | | |
| | imaging | | | | | |
| SEAM | Only detect | TOF-SIMS | 1-μm | No true cell | Nucleus | Small cell size |
| | nuclear | | resolution | types | | |
| | metabolites | | | | | |
| Raman | Only detects | Microscopy | 300-nm | No true cell | Cytosol | Small cell size |
| Spectrometry | limited | | resolution | types | and | |
| | metabolic | | | | nucleus | |
| | targets | | | | | |

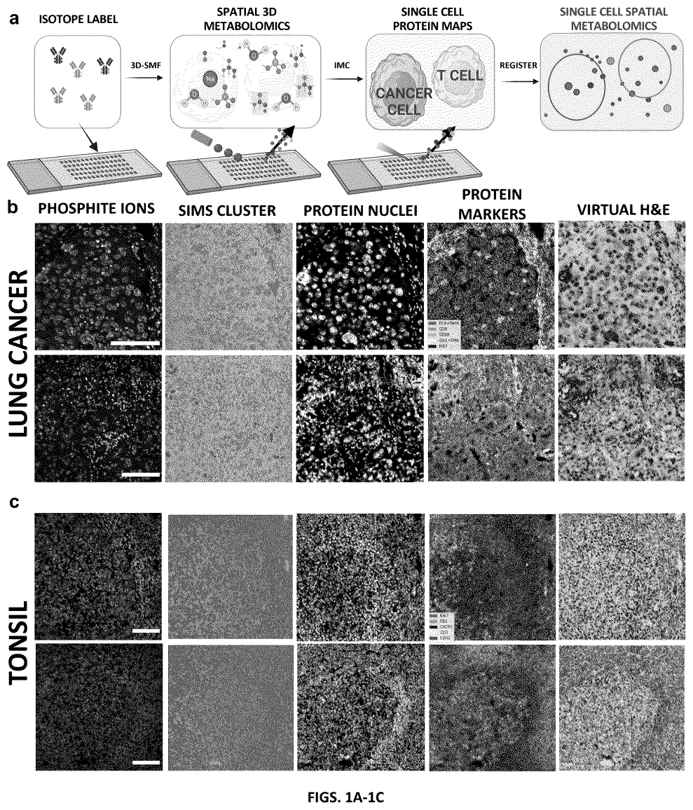

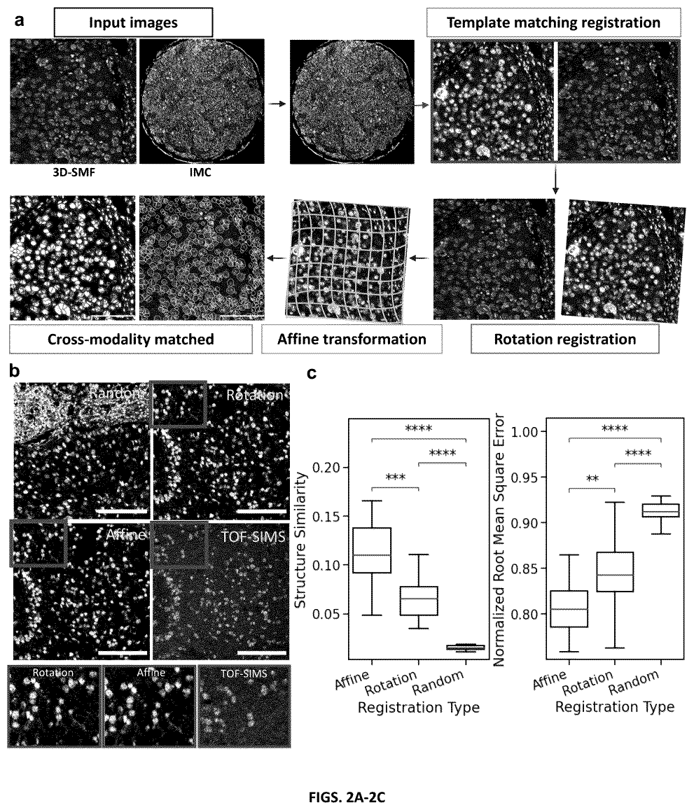

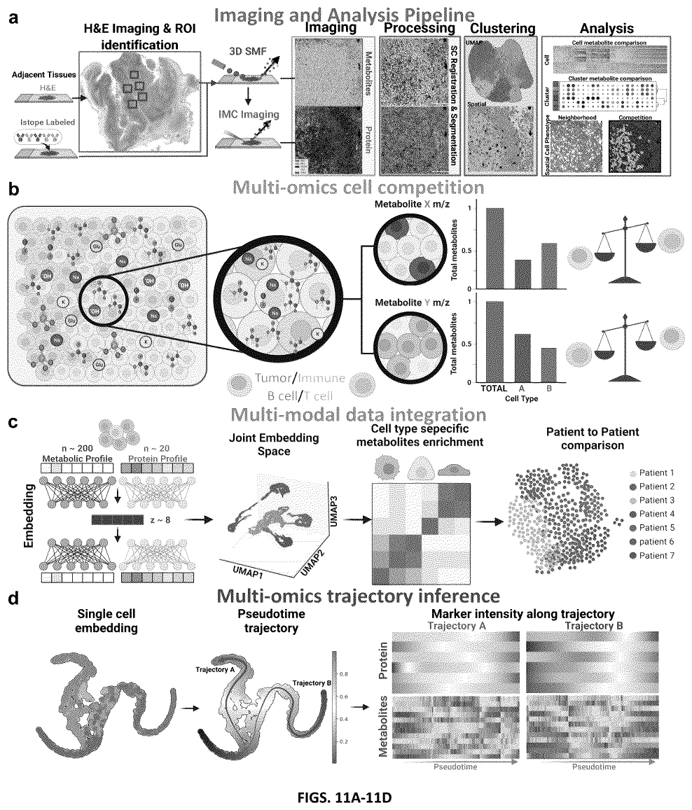

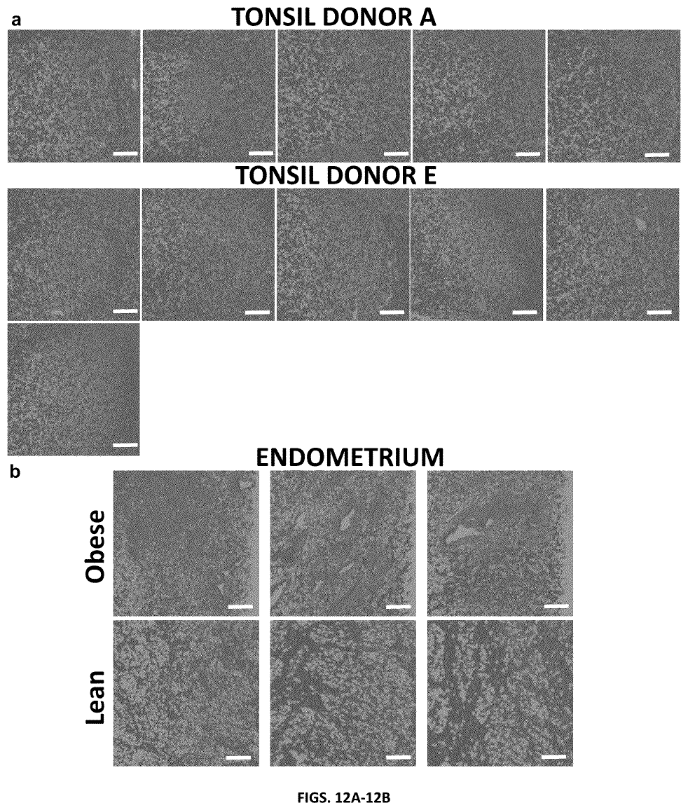

In the ScSpaMet pipeline, the sequential ToF-SIMS and IMC datasets were combined and matched to the single cell level to integrate the information and perform comparative analysis (FIG. 1A). scSpaMet was used to understand the metabolism in lung tumors (FIG. 1B) and tonsil tissues (FIG. 1C). First, a consecutive tissue slide is stained separately using H & E to identify imaging region of interests before scSpaMet profiling and downstream analysis (FIG. 11A). Next, sequential ToF-SIMS and IMC imaging are performed to extract spatial maps of metabolites and proteins. Pixel clustering of SIMS data reveals unique metabolite variation in the spatial context (FIGS. 12A-12B). To quantify cell-type specific metabolite profiles, a cross-modality single-cell registration pipeline was developed utilizing Histone 3 and Intercalator markers in the IMC dataset, and Phosphate 79 m/z channels in TOF-SIMS dataset allowing joint protein-metabolite modalities single cell analysis (FIGS. 2A-2C). Using affine transformation, the cross-modality pipeline yields higher structure similarity and normalized root mean square error compared to rotation only and random shift. The registration quality was quantified using structure similarity and normalized root mean square error. Single-cell segmentation was used to extract the protein and metabolite expression levels and their spatial locations.

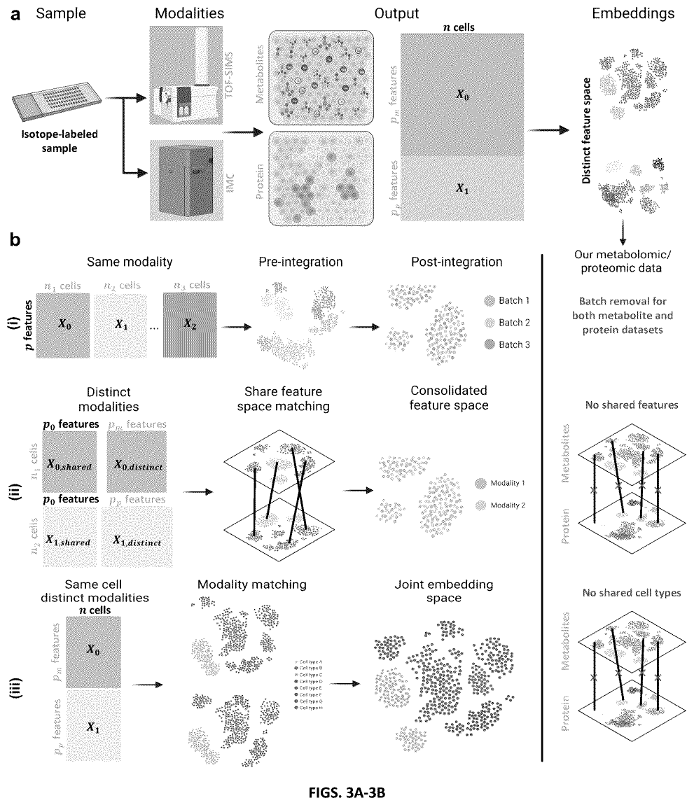

Due to the untargeted discovery nature and low variability nature of metabolomic data (FIG. 3A), is important to develop and identify suitable analysis tools. Existing computational pipelines for integrative analysis of single-cell data are developed for (1) same-modality integration¹⁹⁻²¹, (2) cross-modality integration with shared features^{22,23}, (3) cross-modality multi-modal on same cell^{23,24} (FIG. 3B). The first data integration (1) is for the same type of sequencing modality for batch effect removal in the data. This is also applied to each modality in the metabolomic and proteomic data. The second type of algorithm (2) is working under the assumption that multi-omics data share the same marker set to some extent and multi-omics integration is achieved by finding matching cell in the overlapping feature space across data. The third type of analysis work (3) is for sequencing on the same cell under assumption of matching cell types across modality in multimodal analysis. These methods achieve successful integration by looking at shared markers or cell type. The proteomic/metabolomic single-cell data does not share the same modality nor marker set. Moreover, single cell metabolomic profiles are less variant across cell type therefore unsuitable for using algorithm on integration for same cell sharing cell type. (TABLE 4).

TABLE 4
Comparison of emerging single-cell spatial multi-omic analysis.

| | | Require matching | Same cell | Same |
|---|---|---|---|---|
| Method | Integration modality | markers | measurements | tissue |
| MARIO | Protein and transcriptome | Partially shared | No | No |
| | measurements. (CODEX/ | features/markers | | |
| | CITE-seq) | | | |
| MNNs, | Same modality designed for | Total matching | No | No |
| Scanorama, | scRNA-seq batch effect | | | |
| Conos | removal | | | |
| Seurat v4 | Protein, ATAC and RNA | Partial matching cell | Yes | Yes |
| | | type | | |
| Liger | DNA methylation, chromatin | Partial matching cell | No | No |
| | accessibility and RNA | type | | |
| SpaceM | No integration | No matching. Define | Yes | Yes |
| | | cell type using light | | |
| | | microscope | | |
| SEAM | Metabolites and Transcriptome | No matching. Analysis | No | No |
| | | of correlative | | |
| | | expression from | | |
| | | sequential tissues | | |

The ScSpaMet pipeline provides three additional analysis capabilities for the understanding of multimodal single-cell level data combining protein markers and metabolite channels: (1) multi-omics cell competition (FIG. 11B), (2) multi-modal data integration (FIG. 11C) and (3) multi-omics trajectory inference (FIG. 11D). In the multi-omics cell competition pipeline (1), cell type, cell metabolomics profile, and their spatial location was extracted, and local neighboring cell metabolomic profile variation was compared to infer the local competition of metabolite at the single cell level. In the multi-modal data integration pipeline (2), a cross-modality Variational Autoencoder (VAE) pipeline was adopted to integrate proteomics and metabolomic profiles from the same cell. Because of the imbalance of both data variability (low variation in metabolomic data compared to high variation in proteomic data) and size of the modality (25 protein markers compared to more than 200 metabolite channels), VAE was used to detect specific metabolomic states subset from cell type. Finally, in the multi-omics trajectory inference (3) cell proteomics data was first used to reconstruct cell differentiation trajectory in their spatial domain and correlated with metabolomic profile to understand metabolomic pathways along cell trajectories. This sequential approach allows the study of metabolomic variation along cell trajectory defined by proteomic data.

Taking into account the large number of protein markers profiled using IMC, the Leiden algorithm³⁷ was applied for unsupervised clustering of single-cell proteomics data. Leiden algorithm is widely used in unsupervised single-cell expression clustering³⁸. After inspecting the resulting cluster and assigning it to the corresponding cell phenotype, the correlation of cell type with metabolite channels was studied in situ. Moreover, high-resolution in situ imaging data provided critical spatial information at the single-cell level and allowed cell-cell distance information quantification. Currently, most of the spatial omics frameworks study the cell type neighboring frequency³⁰, probability³⁹, and cell-cell interaction⁴⁰ but not in cell competition from a metabolomic aspect. Therefore, a single-cell multi-omics competition framework was developed by comparing neighboring cell type metabolite ratios and as a function of other cell type distances such as endothelial cells. Next, VAE⁴¹ has shown an improved ability to model single-cell data distribution in both proteomics²⁷ and transcriptomics²⁵ with the ability to integrate different modalities and therefore was adopted here to extract joint latent representation from proteomic and metabolomic data. Finally, single-cell trajectories have largely been limited to RNA-based technologies⁴² due to higher throughput gene sequencing and proteomic trajectory analysis for well-defined cell lineages⁴³. Here, single-cell trajectories were reconstructed with diffusion pseudotime analysis²⁹ for well know the developmental progression of B-cells in human tonsil follicles characterized by their protein expression.

Discussion: Spatially resolved metabolomic analysis of human tissues is paramount for the study of chemical balances and alterations in health and disease. Metabolites and lipids play a regulatory role in immune responses and cancer^{1,2}. Particularly, metabolism in tumors has demonstrated vital mechanisms in understanding the functional changes in immune and cancer cell interactions³. Immune cell types experience significant metabolic programming when infiltrating the tumor ecosystem⁴⁻⁶. Cancer progression controls multiple immune and stromal cell types and their metabolic functional roles in tumors due to the rapid nutrient depletion and accumulation of waste products during rapid proliferation⁷. Thus, it is crucial to identify cell types and their metabolism in biological tissues.

Recent advances in Mass spectrometry imaging (MSI) techniques have allowed spatial profiling of a large number of proteins and metabolites. MSI methods have provided capabilities to capture metabolite information within its spatial context to address the loss of spatial details in the bulk-level mass spectrometry techniques⁸. Several technological advancements in mass spectrometry imaging have allowed the acquisition of spatial metabolite features including matrix-assisted laser desorption/ionization (MALDI), desorption electrospray ionization (DESI), and secondary ion mass spectrometry (SIMS)⁹. MSI methods characterized metabolic heterogeneity in tissue samples with different sensitivity, spatial resolution, and chemical coverage. MALDI and DESI efficiently map metabolites at 20-100 μm spatial resolution while TOF-SIMS acquires lipids at sub-micro spatial details¹⁰⁻¹². However, these MSI approaches lacked cell type tagging, causing the loss of key cell-specific correlates in tissues.

Characterization of the single-cell level metabolomic profile remains a difficult task. A recently proposed method called SpaceM¹³ performed an integrated analysis of MALDI-based metabolic maps and fluorescence microscopy images in single cultured cells but lacked high-resolution details of “true” single cells in dense tissues. Instead, SpaceM mapped out pixelated correlations of metabolic targets within cytosolic boundaries of large cells in cultures. Another method called SEAM¹⁴ was proposed for single nuclear metabolomic profiling in the native tissue microenvironment using TOF-SIMS. While this method has allowed submicron metabolite mapping in cells and tissues, the association of specific cell types to the metabolic profiles has been lacking and only nuclei patterns are extracted from the entire cells without the cytosolic boundaries. On the other hand, Imaging Mass Cytometry (IMC) has provided multiplex imaging of 35 protein markers in the spatial context of patients' samples at subcellular resolution (1-μm)¹⁵⁻¹⁷, albeit without metabolic target information. Recently, a three-dimensional (3D) spatially resolved metabolic profiling framework (3D-SMF)¹⁸ was demonstrated to incorporate isotope tagging of cell-type specific channels measured by TOF-SIMS. While this method showed promising results for detecting correlations of cell types with metabolic channels, the capability of achieving single-cell details of joint protein and metabolite analysis has not fully been realized in spatially crowded human tissues.

Several algorithms have been developed for the integration of multi-omics data such as MNN¹⁹, Scanorama²⁰, Conos²¹, MARIO²², Seurat²³, LIGER²⁴, and SEAM¹⁴. MNN, Scarnorama, and Conos provide computational pipelines for batch effect removal from multiple datasets. MARIO integrates multi-omics datasets by matching with partial overlap accounting for both shared and distinct features. Seurat uses an unsupervised framework to integrate multi-omics datasets by assigning relative weights of each data type in each cell. LIGER formulates an integrative nonnegative matrix factorization problem to address multi-omics data integration from different modalities and protocols. While multi-omics integration methods are being developed, spatial metabolomic data are unsuitable for integration with other modalities because of their untargeted nature, lack of shared markers, and identified cell types. On the other hand, single-cell analysis methods such as VEGA²⁵, scDHA²⁶, scMM²⁷, SPADE²⁸, DPT²⁹, Squidpy³⁰, Athena³¹, and SPEX³² were developed for latent embedding generation, cell lineage reconstruction, and spatial analysis. Using an autoencoder model, VEGA, scDHA, and scMM proposed methods for extracting single-cell level latent variables suitable for downstream analysis such as clustering and visualization. SPADE and DPT are methods developed for inferring single-cell developmental progress from data. Squidpy, Athena, and SPEX are a suite of algorithms for analyzing spatial omics data by introducing graph construction from single-cell spatial data and characterization of cell type neighboring frequency and spatial properties. While those methods showed great progress in the analysis of proteomics data, they are not tailored for single-cell metabolomics and no work is focusing on single-cell competition^{33,34}. It is therefore important to introduce specific analysis pipelines adapted for joint metabolomic and proteomic datasets.

To provide a complementary solution to the need for simultaneous whole-cell metabolic and protein analysis in situ, Single Cell SPAtially resolved METabolic (scSpaMet) framework is proposed herein for profiling immune cells and cancer cells in human tissues at the single-cell level by incorporating untargeted spatial metabolomics and targeted multiplexed protein imaging in a single pipeline. scSpaMet combines previously developed 3D-SMF¹⁸ submicron resolution metabolites imaging framework with multiplex IMC proteomic imaging for cell-type characterization in the same tissue sample. scSpaMet enables the correlation of more than 200 metabolic markers and 25 protein markers in individual cells within native tissues. Moreover, scSpaMet introduces additional analysis capabilities for joint metabolomic and proteomic single-cell data.

ScSpaMet is a framework for joint protein-metabolite imaging at the single-cell level. ScSpaMet framework allows systematic single-cell segmentation, phenotyping from protein data, and metabolite profiling on the same tissues while providing high spatial resolution. While 3D-SMF profiling captures incomplete molecule fragments⁷⁶, it produces highly multiplex metabolite imaging and minimal sample alteration allowing further correlative imaging from IMC. The segmentation and analysis of single-cell protein and metabolic feature profiles can be performed directly in their tissue sections. scSpaMet can be applied to human lung cancer, tonsil, and endometrium tissues. ScSpaMet identifies metabolite variation between cells in the tumor and stromal regions, cell type-specific local metabolic competition, metabolic trajectories, and patient-level molecule variation in lung cancer tissues. Similarly, scSpaMet quantifies metabolic changes around B-cell follicles in human tonsil tissues by looking at B-cells, T-cells, and FDCs inside germinal center LZ/DZ regions, cell type-specific local metabolic competition inside of germinal center, and metabolomic changes along B-cell differentiation trajectories. Finally, human endometrium tissues can be imaged using scSpaMet to decipher the metabolic composition of cell type and comparison between lean and obese conditions.

One potential limitation is the challenge in metabolite annotation and coverage for lipids, amino acids, and the metabolism pathway from TOF-SIMS. Another limitation is the relatively small imaged regions from tissue samples. This is a trade-off between imaging speed and spatial resolution in the TOF-SIMS. But multiplexed protein images from IMC are able to discern the heterogeneity of the tumor microenvironment. The immune panel can be expanded to analyze further phenotypes such as macrophage M1/M2 or B-cells subtypes. OCT-embedded frozen tissues also showed better metabolite preservation compared to FFPE samples, but the tissue structures are less preserved therefore the need to incorporate cryo-imaging into the pipeline. Finally, another aspect is the lack of information related to the treatment history of the patients as it can alter the metabolite state within the tumors and help explain metabolite variation across patients.

Despite these limitations, scSpaMet provides a complementary solution to the need for simultaneous whole-cell metabolic and protein analysis in situ by incorporating untargeted spatial metabolomics and targeted multiplexed protein imaging in a single pipeline. scSpaMet registers single-cell measurements in bi-modality and enabled accurate identification of various cell types with their corresponding metabolomic profiles.

In summary, scSpaMet allows high-resolution joint protein and metabolite profiling at the single-cell level in the same tissue. Using protein markers, single cells are annotated and their metabolic variation is quantified. Combining cell type spatial information and metabolic profile, a local cell metabolite competition framework is proposed. Moreover, metabolic reprogramming along cell differentiation trajectories can be retraced and projected spatially. With the advancement of spatial mass spectrometry imaging resolution, molecule annotation capabilities, and efficiency, scSpaMet paves the way to systematic single-cell metabolite and protein profiling in their tissue environment.

### Example 3—Cell Type-Specific Metabolic States in Lung Cancer Tissues

Cancer cells can change their metabolic programming to meet their increasing energy needs for rapid progression and aggressive growth. This results in a tumor microenvironment depleted of critical nutrients, hypoxic, and acidic. These factors can lead to the lower recruitment and activation of the tumor-effector immune cells. It is critical to understand the differential metabolic requirements of the diverse immune cells in response to the growing tumors to uncover novel metabolic relations that can open the door for therapeutic interventions combining chemical perturbations with targeted immunotherapies³.

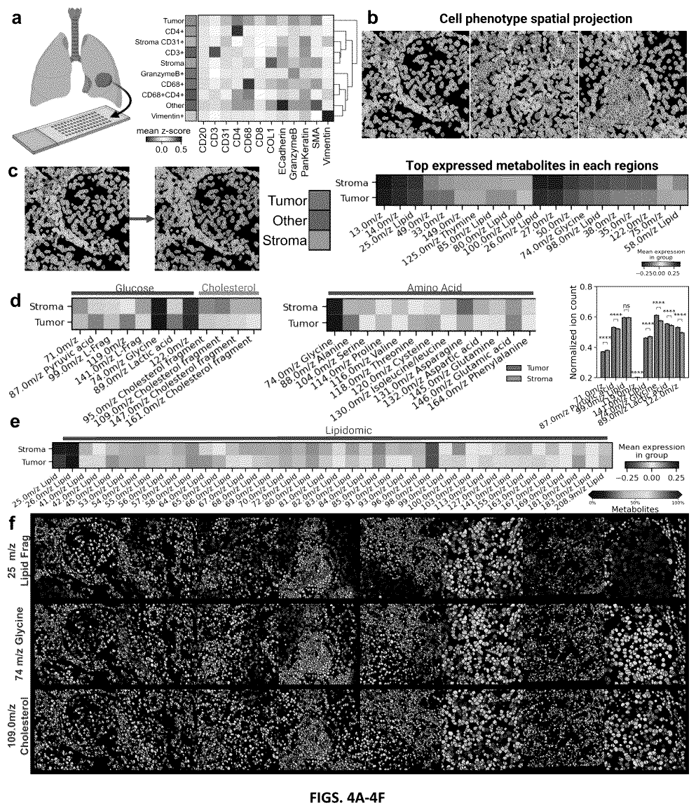

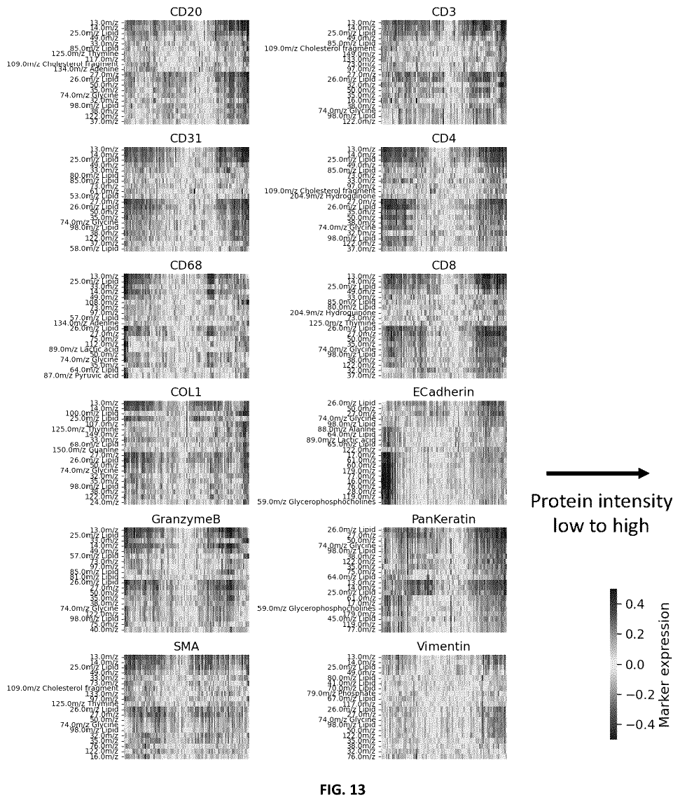

ScSpaMet was applied to a lung tumor microarray for the characterization of the protein-metabolic environment inside the tumor microenvironment (FIG. 13). A tumor microarray of lung adenocarcinoma (grade III) with the tissue (BioMax, ID: BS04081a) was stained with an antibody panel of 21 markers (TABLE 5) spanning tumor, stromal, endothelial, and immune markers. ScSpaMet first identified single-cell protein phenotypes from the isotope-conjugated antibody panel of IMC using the Leiden algorithm to find cluster of cells (n=10) with similar expression profiles and annotated clusters by looking at their mean expression (FIG. 4A). Cancerous/paracancerous regions were labeled with clusters containing high expression level of pan-keratin and e-cadherin whereas stromal regions were marked by the expression of smooth muscle actin (SMA) and collagen type 1 (COL1). Immune cells were extracted based on the expression of specific protein markers (CD4, CD68, CD3). The representative patients' tissue images were then reconstructed from the cell masks and their clustering results by assigning each segmented cell to its corresponding cluster (FIG. 4B). In each patient tissue image, single cells (n=19507) were then classified into tumor and stroma regions based on their protein phenotypes and spatial localization enabling comparison of highly expressed metabolites at the single cell level in each of the two regions (FIG. 4C).

TABLE 5
List of antibodies, their conjugated metal tags,
their clones, and their concentrations used for
imaging mass cytometry in lung cancer microarrays.

| Marker | Clone | Metal Tag | Dilution |
|---|---|---|---|
| CD20 | H1 | 161Dy | 1:400 |
| CD3 | Polyclonal | 170Er | 1:100 |
| CD4 | ERP6855 | 156Gd | 1:200 |
| CD45RO | UCHL1 | 173Yb | 1:50 |
| CD68 | KP1 | 159Tb | 1:50 |
| CD8a | C8/144B | 162Dy | 1:100 |
| FoxP3 | 236A/E7 | 155Gd | 1:30 |
| Pan-Keratin | C11 | 148Nd | 1:100 |
| Granzyme B | EPR20129-17 | 167Er | 1:100 |
| Ki-67 | B56 | 168Er | 1:50 |
| SMA | 1A4 | 141Pr | 1:200 |
| Collagen type I | Polyclonal | 169Tm | 1:300 |
| E-cadherin | 24E10 | 158Gd | 1:50 |
| Histone 3 | D1H2 | 171Yb | 1:50 |
| Vimentin | D21H3 | 143Nd | 1:100 |
| CD11b | EMP1344 | 149Sm | 1:50 |
| CD44 | IM7 | 153Eu | 1:100 |
| CD31 | EPR3094 | 151Eu | 1:100 |
| CD45RA | HI100 | 166Er | 1:50 |
| CD11c | Polyclonal | 154Sm | 1:100 |
| Intercalator | — | 191Ir/193Ir | 1:400 |

The ScSpaMet captures the products of glycolysis metabolism instead of directly profiling glucose-associated small molecules due to inefficient detection by the TOF-SIMS instrument. Cancer cells upregulate the glycolytic catabolism of glucose into lactate even under normoxia. That leads to elevated levels of lactate, adenosine, kynurenine, ornithine, reactive oxygen species (ROS), and potassium, contributing to the suppression of an anti-tumor response. Mass channels characterized by mass-to-charge ratios (M/z) were obtained from scSpaMet and correlated with known annotation from literature search and categorized the corresponding metabolite peaks into glucose, cholesterol, amino acid, and lipid fragments (See Methods). Selected mass channels of 74.0 m/z Glycine, 89.0 m/z Lactaid acids, and 122.0 m/z Adenine related to glucose metabolism have higher expression in tumor regions (FIG. 4D)). On the other hand, Cholesterol fragmentation related channels show higher cholesterol expression levels in stromal regions compared to tumor regions. Cholesterol is one of the most essential lipids for the cells' development, but cancer cells show more rapid depletion of the cholesterol than normal cells indicating their uncontrolled proliferation⁴⁴. For identified amino acid and lipid-related channels, the single-cell expression levels inside stroma and tumor regions exhibit high variability (FIG. 4E). Single-cell metabolite spatial maps were reconstructed to visualize metabolite variation across regions in patients' tissue images. The 25 m/z lipid, 74 m/z Glycine, and 109 m/z Cholesterol fragments were shown in their spatial localization with correlation to define tumor and stromal regions (FIG. 4F).

### Example 4—Cell Neighborhood Metabolite Competition from Vessels

Metabolism reprogramming occurs in tumor and non-tumor components of the tumor microenvironment (TME) by metabolic competition around tumor cells for a steady supply of nutrients even under hypoxic conditions⁴⁵. Endothelial cells inside the lining of the vascular system play essential roles in the tumor microenvironment for promoting or preventing tumor progression⁴⁶ to support tumor metabolism⁴⁷ and metabolic reprogramming4.

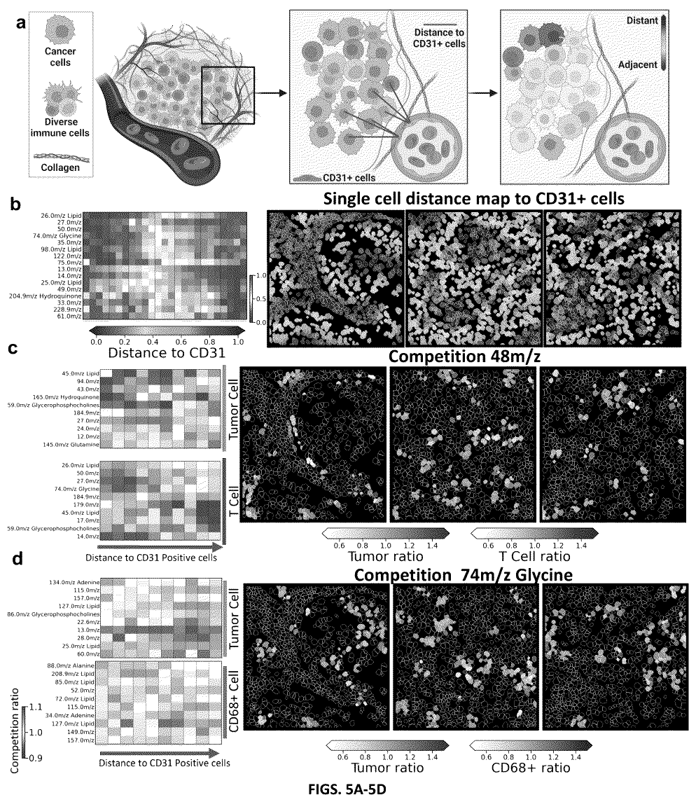

To study the impact of nutrient delivery around vascularization sites on chemical regulation, a framework for single-cell local metabolite competition analysis in the lung tumor microenvironment was developed. First, by leveraging CD31 protein markers from IMC multiplex data, CD31+ endothelial cells were defined in each patient tissue image. To consider the small size of the imaging field of view (FOV) in the 3D-SMF pipeline, the whole TMA core IMC images were used to detect CD31-positive cells. Single-cell CD31 intensity expression followed a bi-modal Gaussian distribution and cells with higher intensities were defined as CD31+ endothelial cells and validated by inspecting original CD31 marker images. Each 3D-SMF image region was matched back into the IMC images. Then for each segmented cell in the 3D-SMF image region, the minimum distance to CD31+ endothelial cells was extracted by identifying the cell centroids' position closest to the CD31+ endothelial cells from larger IMC CD31 images by kNN search with spatial data of single-cells (FIG. 5A). In each tissue sample, the distance to CD31+ cells was used to generate metabolomic gradients (normalized in the range of 0 to 1) from distance maps by binning distance into 20 bins and averaging cell metabolite expression per bins (FIG. 5B). Specific lipid channels such as 25.0 m/z, 49.0 m/z, and 33.0 m/z were up-regulated around the CD31+ endothelial cells whereas 26.0 m/z, 74.0 m/z, and 98.0 m/z were up-regulated further away from CD31+ endothelial cells.

Neighboring cells in the tumor microenvironment enter a local nutrient competition due to the high proliferation nature of tumor cells and their need for nutrients. Here, the metabolite ratio of neighboring T- and tumor cells was quantified by modeling the local metabolite competition of tumor and CD3+ T-cells as a distance of CD31+ endothelial cells (FIG. 5C). First, a single-cell neighboring map was constructed from single-cell spatial data by specifying a radius of 20 μm. For each cell, the metabolite competition ratio was defined as the metabolite expression of the cell divided by the median metabolite expression of its neighboring cells (ratio of 1 would mean equal metabolite level between a cell and its neighbors). Finally, the analysis of cell competition was combined with distance to CD31+ endothelial cells. The mass channels of 148.0 m/z Methionine, 42.0 m/z lipid, 94.0 m/z, and 45 m/z lipid fragments have higher expression in tumor cells compared to T-cells at a further distance from CD31+ endothelial cells. On the other hand, the mass channels of 145.0 m/z Glutamine, 48.0 m/z, and 27.0 m/z exhibited higher expression in tumor cells compared to T-cells in the proximity of CD31+ endothelial cells. For T-cells, 148.0 m/z Methionine, 42.0, 81.0, and 100.0 m/z lipid fragments were upregulated in close proximity to CD31+ cells. scSpaMet reconstructed the metabolite competition maps of neighboring cells as a function of relative distances to the CD31+ endothelial cells overlaid on a spatial metabolomics map of the tissue.

Similarly, the tumor and CD68+ cells' local metabolite competition was analyzed as a distance from CD31+ endothelial cells (FIG. 5D). The mass channels of 129.0 m/z, 134.0 m/z Adenine, and 126.0 m/z Glycerophosphore demonstrated higher expression in tumor cells compared to CD68+ cells at a further distance from CD31+ endothelial cells. On the other hand, the mass channels of 165.0 m/z Hydroquinone, 41.0, 42.0, 167.0 m/z lipid fragments, and 131 Asparagine exhibited higher expression in tumor cells compared to CD68+ cells in the proximity of CD31+ endothelial cells. For CD68+ cells, only the mass channel of 208.9 m/z lipid fragment was upregulated further from CD31+ cells. The mass channels of 61.0, 60.0, 77.0, and 28.0 m/z yielded high expression in CD68+ cells compared to tumor cells consistently. In the proximity of CD31+ endothelial cells, the mass channels of 134.0 m/z Adenine, 118.0 m/z Threonine, and 144.0 m/z were upregulated in CD68+ cells. The corresponding spatial map of local metabolite competition was visualized for cancer and CD68+ cells.

### Example 5—Patient-Level Spatial Metabolomics Difference in Lung Cancer

Lung cancer is heterogeneous not only at a cellular level⁴⁸ but also at the patient level with implications in the understanding of pathogenesis, diagnosis, and personalized therapy⁴⁹. It is therefore imperative to decode the patient-to-patient variability of protein and metabolite distributions in single cells from different tumor biospecimens. ScSpaMet pipeline enabled quantification and comparison of metabolite profile at the patient level in lung cancer data. For each patient, scSpaMet was used to image 3 regions of interest per tumor. The patient-level metabolite distribution was then extracted and compared across patients.

Patient-level metabolite distributions were stratified into high-variation and low-variation metabolite channels (TABLE 6). By comparing patient-level metabolite distribution, lipid fragment was distributed between high and low-variation channels. Cholesterol fragment channels (95.0 m/z and 147.0 m/z) indicated low variation across patients. Glucose pathway-related channels such as 71.0 m/z, 87.0 m/z pyruvic acids, 89.0 m/z lactic acid, and 119.0 m/z demonstrated considerable variation across samples. Next, the variability of single-cell competition for tumor/T-cells and tumor/CD68+ cells was examined for annotated metabolite channels. For T-cells and CD68+ cells, the mean competition expression was plotted for metabolite channels corresponding to glucose, cholesterol, amino acid, and lipid fragments.

TABLE 6
BS04081a patient specifications for cancer type, stage, and grade.

| | | Pathology | | | |
|---|---|---|---|---|---|
| Core number | Sex | Diagnosis | Grade | Stage | Type |
| B5, F7 | M | Adenocarcinoma | 3 | IIIA | Malignant |
| C6, F4 | M | Adenocarcinoma | 3 | IIA | Malignant |
| D4, E4, E6 | M | Adenocarcinoma | 3 | IB | Malignant |

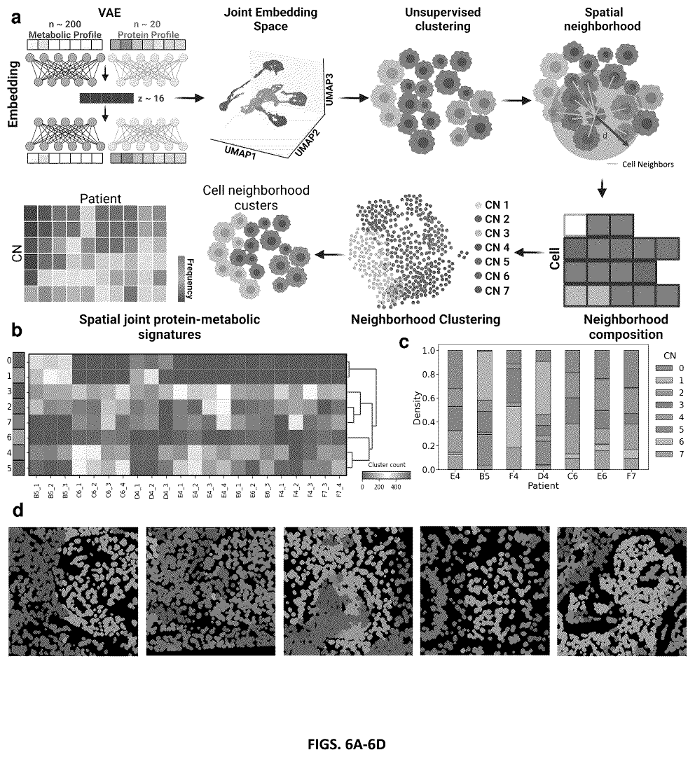

To enable robust comparisons across metabolic and proteomic profiles of distinct patients, scSpaMet analyzed joint protein-metabolite maps with a Variational Autoencoder (VAE) architecture. The VAE takes as input both the metabolite and protein profile of single cells and outputs the reconstructed profiles, that is a vector of size 21 for protein data and size 8 from each modality. The resulting latent space embedding is used for clustering and showed multimodal phenotyping showing distinct metabolite states in tumor and stromal cell types. By stratifying single-cell bi-modal spatial maps using self-supervised learning, the intra- and inter-patient variability was uncovered at the imaged region size 198 for metabolite data. The embedding layer of the VAE was used to capture a latent variable of the joint protein-metabolite profile at the single-cell level by combining a latent vector of interest level. Moreover, spatial joint metabolic-proteomic signatures were examined for different groups of patients. In order to generate spatial signatures incorporating joint metabolomic and proteomics levels, unsupervised clustering labels were obtained for each cell based on their VAE latent embedding, and each cell neighboring information was extracted by taking a radius threshold of 25 μm. For each cell, the unsupervised cluster labels in its neighborhood were counted, and a vector corresponding to the count of each clustering type around its neighborhood was obtained. After normalization of the vectors by their total count (with a density equal to one per cell), a second unsupervised clustering was performed which takes into account the spatial signature of joint metabolic and proteomics profiles (FIG. 6A). The intra- and inter-patient spatial signature variability was uncovered in each region of interest (FIGS. 6C-6D).

### Example 6—Cell Type-Specific Metabolic States Around B Cell Follicles in Tonsil Tissues

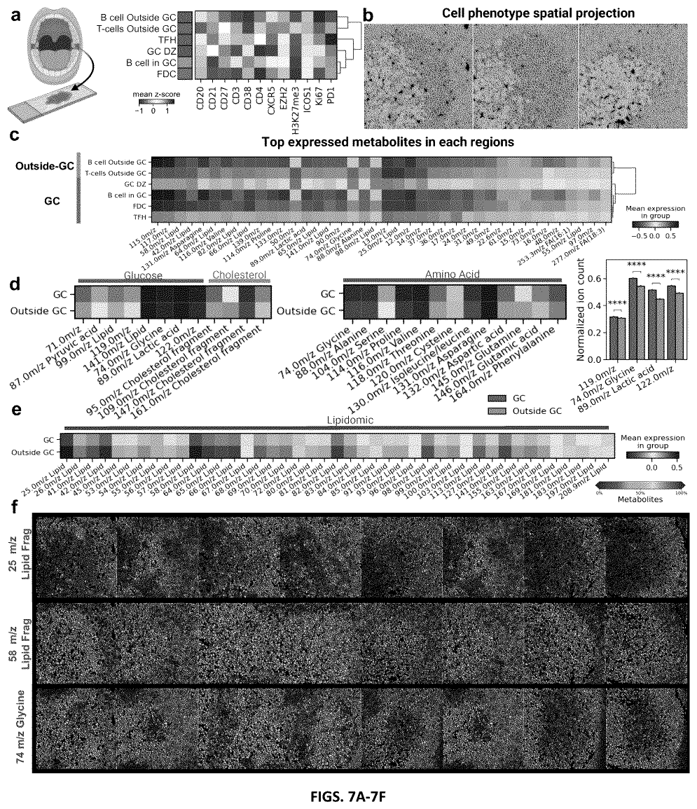

Tonsils play an important role in the immune system and are part of the secondary lymphoid organs. They are composed predominantly of B- and T-cells populations in coordination with other immune cells and epithelial cells around the tonsil follicles regions^{35,50,51}. It was reasoned that deciphering spatially resolved cellular composition around B-cell follicles and how metabolite variations occur within B-cell subsets would be valuable for decoding the role of chemical balance in humoral immunity. ScSpaMet was applied to healthy human tonsil tissues (TABLE 7) to characterize the protein-metabolic environment around B-cell follicles. Herein, an antibody panel of 25 markers included immune surface markers, cytokine markers, epigenetic regulators, and extracellular matrix proteins (TABLE 8). ScSpaMet first identified single-cell protein phenotypes (n=6) using the Leiden algorithm (FIG. 7A) and representative patients' tissue images were then reconstructed from the cell masks, and their clustering results by assigning each segmented cell to its corresponding cluster (FIG. 7B). In each patient tissue image, single-cell phenotypes (n=31156) from unsupervised clustering were classified into the follicle zone, outside follicle zone, germinal center light zone and germinal center dark zone defining inside and outside GC regions enabling comparison of highly expressed metabolites in each region (FIG. 7C).

TABLE 7
HuFPT161 patient specifications for tonsil tissue.

| Donor No. | Sex | Pathology Diagnosis |
|---|---|---|
| A-E | M | Normal Tonsil Tissue |

TABLE 8
List of antibodies, their conjugated metal tags, their clones, and their
concentrations used for the imaging mass cytometry tissue labeling step.

| | | | | Antibody | |
|---|---|---|---|---|---|
| | | | | Stock | |
| | Isotope | | | Concentration | Experimental |
| Marker | Metal Tag | Clone | Vendor | (mg/ml) | Dilution |
| CD38 | 141Pr | EPR4106 | Standards Biotools | 0.5 | 1:100 |
| Vimentin | 143Nd | D21H3 | Standards Biotools | 0.5 | 1:100 |
| BCL6 | 147Sm | K112-91 | Standards Biotools | 0.5 | 1:50 |
| ICOS1 | 148Nd | D1K2T | Standards Biotools | 0.5 | 1:50 |
| CD11B | 149Sm | EPR1344 | Standards Biotools | 0.5 | 1:100 |
| CD11C | 154Sm | Polyclonal | Standards Biotools | 0.5 | 1:50 |
| FOXP3 | 155Gd | 236A/E7 | Standards Biotools | 0.5 | 1:50 |
| CD4 | 156Gd | ERP6855 | Standards Biotools | 0.5 | 1:200 |
| CD20 | 161Dy | H1 | Standards Biotools | 0.5 | 1:400 |
| CD8a | 162Dy | C8/144B | Standards Biotools | 0.5 | 1:100 |
| C-Myc | 164Dy | 9E10 | Standards Biotools | 0.5 | 1:100 |
| PD1 | 165Ho | ERP4877(2) | Standards Biotools | 0.5 | 1:50 |
| Ki67 | 168Er | B56 | Standards Biotools | 0.5 | 1:50 |
| Collagen | 169Tm | Poly | Standards Biotools | 0.5 | 1:300 |
| type I | | | | | |
| CD3 | 170Er | Poly | Standards Biotools | 0.5 | 1:100 |
| CD27 | 171Yb | EPR8569 | Standards Biotools | 0.5 | 1:100 |
| CD138 | 158Gd | EPR6454 | *Abcam | 0.62 | 1:2000 |
| CXCR5 | 159Tb | EPR23463-30 | *Abcam | 0.47 | 1:500 |
| EZH2 | 173Yb | EPR9307(2) | *Abcam | 0.49 | 1:120 |
| CD21 | 145Nd | EP3093 | *Abcam | 0.48 | 1:120 |
| CD86 | 151Eu | BU63 | *Abcam | 0.65 | 1:300 |
| CD83 | 167Er | EPR23809-19 | *Abcam | 0.37 | 1:200 |
| CXCR4 | 153Eu | UMB2 | *Abcam | 0.73 | 1:600 |
| H3K27ME3 | 176Yb | C36B11 | *Cell Signaling | 0.49 | 1:120 |
| | | | Technology | | |
| Nucleic | 191Ir/193Ir | n/a | Standards Biotools | 125 μM | 1:400 |
| Acid | | | | | |

*refers to the markers/antibodies with in-house conjugations to the metal tags.

The inside and outside germinal center regions demonstrated statistically significant metabolite distribution variation and the mass channels obtained from scSpaMet were correlated with metabolite peaks annotated as glucose, cholesterol, amino acid, and lipid fragments. Rapid proliferation is key to affinity maturation, but little is known about how GC B cells fulfill the metabolic demands required to achieve the GC reactions. In lymphoid organs, B cells inside the GC undergo changes that lead to increased glucose consumptions⁵². When B cells get stimulated through their B cell receptor (BCR) or the costimulatory protein CD40 or the Toll-like receptors (TLRs), the Hypoxia-inducible factor-1 (HIF-1) and the c-Myc expression are enhanced, leading to higher oxygen consumption, enhanced glycolysis, and increased production of lactate. This process causes higher consumption of amino acids including alanine and glutamine used as carbon and energy sources. Recent evidence suggested that GC B cells obtained the required energy from the fatty acid oxidation (FAO) pathway⁵³⁻⁵⁵. This finding was counterintuitive because other highly proliferative B cell blasts still exhibited high glycolysis activity, but GC B cells upregulated FAO while performing minimal glycolysis. Inside the GC regions, scSpaMet provided overall higher metabolite expression related to glucose fragments (71.0, 87.0, 99.0, 119.0, 141.0 m/z), glucose pathway fragment (74.0 m/z Glycine, 89.0 m/z Lactic acids), cholesterol fragment channels, and amino acid-related channels but selected Fatty Acid channels (253.3 m/z and 277.0 m/z) showed higher expression outside of GC (FIGS. 7C-7D). Analysis of lipid-related channels demonstrated up-regulation in lipid fragments inside GC compared to outside GC (FIG. 7E). The mass channels of 25.0 m/z lipid fragment, 58.0 m/z lipid fragment, and 74 m/z Glycine fragment were shown in their spatial localization with correlation to distinct GC regions (FIG. 7F). VAE architecture was also used for joint protein-metabolite embedding. The result showed unique metabolites states inside and outside of germinal centers.

### Example 7—Single-Cell Metabolite Local Competition Around Germinal Centers

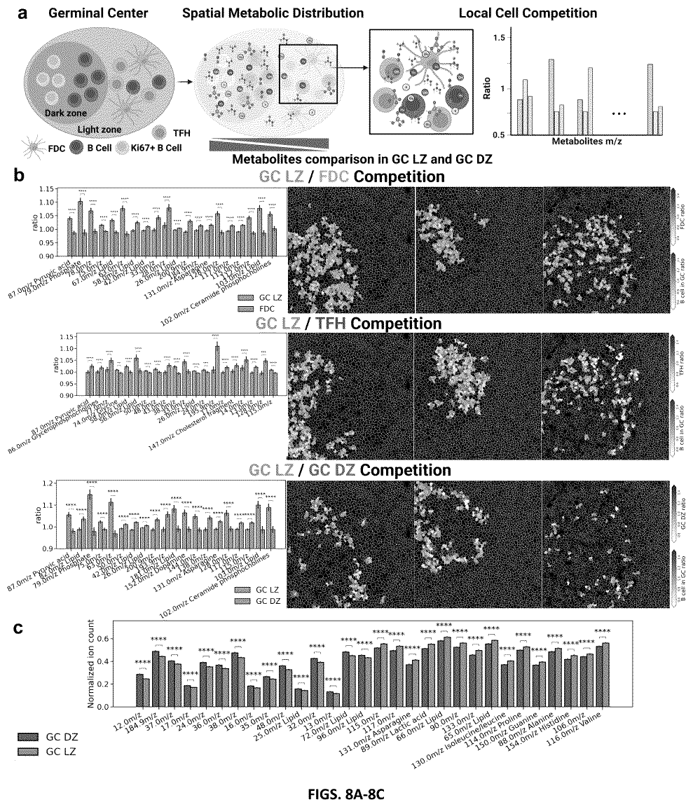

Humoral immunity against infections depends on the germinal center (GC) differentiation process in the B cell follicles of secondary lymphoid organs. In GCs, naïve B cells rapidly proliferate in response to T cell-dependent antigens and somatically mutate into high-affinity antibody-secreting cells, i.e., plasma cells⁵⁶. In this GC process, B cells rapidly alternate between distinct “metabolic states” across quiescence, proliferation, and differentiation⁵⁷. Rapid proliferation is key to affinity maturation, but little is known about how GC B cells fulfill the metabolic demands required to achieve the GC reactions. ScSpaMet enables the characterization of neighboring cell-to-cell local metabolite competition inside the GC. Single cells were phenotyped inside the tonsil germinal centers using cell protein profiles to determine GC B-cells, T-cell follicular helper cells (TFHs), and Follicular dendritic cells (FDCs) with their corresponding metabolites distribution in their spatial environment. Similarly to the lung cancer competition pipeline, per-cell local cell competition metabolite ratios were calculated as the metabolite expression of the cell divided by the average metabolite expression of its neighboring cells. (FIGS. 8A-8B). In human tonsil tissues, because of the high density of single cells in germinal centers, local cells were defined as in competition when the single cell masks shared a boundary.

B-cell tonsil GCs are polarized into light (LZ) and dark (DZ) zones with functional and phenotypic distinction at the single-cell level⁵⁸. Ki67 is a protein marker for the characterization of cell proliferation⁵⁹. ScSpaMet identified GC LZ and DZ using Ki67, CD20, CD21, CD38, and EZH2 markers (FIG. 8C), and their metabolic distribution was compared. GC LZ exhibited a higher expression of 89.0 m/z lactic acid, 88.0 m/z Alanine, and other amino acid channels.

### Example 8—Metabolic Trajectory Analysis of Pseudotime B Cell Differentiation

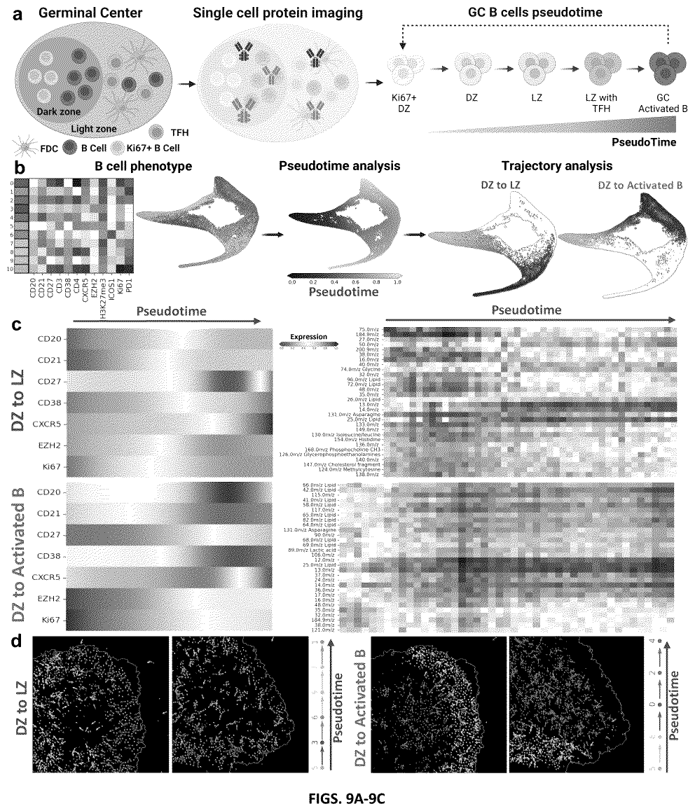

GCs of lymphoid organs are the place where activated B-cells undergo differentiation across Dark Zone (DZ) B-cells, Light Zone (LZ) B cells, Memory B-cells, and Plasma cells⁶⁰⁻⁶⁰. DZ contains the rapidly dividing B-cells undergoing somatic hypermutation (SHM) and LZ contains FDCs, TFH, and B-cells that are exiting the GC area. B-cell migration happens inside GC from DZ to LZ⁶³. Moreover, recent studies have provided ample experimental evidence for the re-entry of selected B cells from LZ to DZ upon antigen-driven selection Moreover, recent studies have provided ample experimental evidence for the re-entry of selected B cells from LZ to DZ upon antigen-driven selection⁶⁴⁻⁶⁶. B-cell pseudotime analysis inside germinal centers has been used to trace their developmental trajectories using RNA-seq and protein data⁶⁷⁻⁷⁰. ScSpaMet enables B-cell trajectory analysis from single-cell protein expression with metabolite correlation in their spatially resolved tissue coordinates. By incorporating the single-cell protein phenotypes inside tonsil GC, scSpaMet infers the pseudotime trajectory of the GC B-cells population (FIG. 9A).

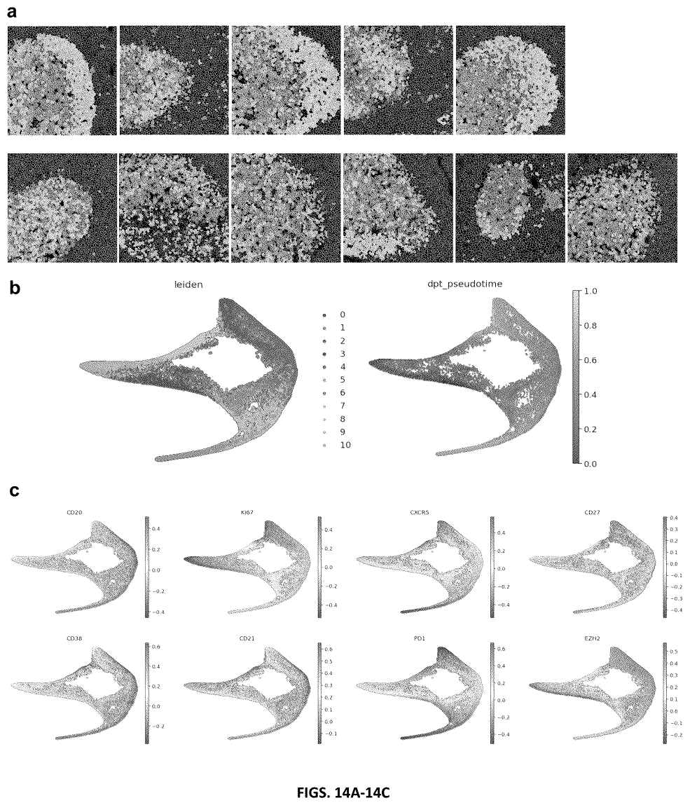

Using multiplexed protein markers, B-cells inside of GC (n=15655) were selected for unsupervised phenotyping with the Leiden algorithm (FIG. 14A), projected into their t-distributed stochastic neighbor (t-SNE) embedding space, and their pseudotime ordering was inferred by determining the cell state and calculating the respective probability of differentiating into each terminal state⁷¹ (FIGS. 14B-14C). This process computationally reconstructed two different differentiation trajectories of B cells inside GC (FIG. 9B). Here single-cell hierarchy was inferred from their protein profiles. The starting point was selected from single-cell expression corresponding to Dark Zone B-cells. After plotting the cells in the embedding space (FIG. 9B), two distinct trajectories were determined that represented DZ to LZ and DZ to activated B cells from single-cell protein expression across trajectories and their corresponding diffusion pseudo time value. The bifurcation point was determined empirically by the embedding plot of the single cells. ScSpaMet then characterized the metabolite variations along the defined B-cell trajectories, including the pseudotime path (1) the GC DZ B-cells to GC LZ B-cells and (2) the GC DZ B-cells to the activated B-cells (FIG. 9C).

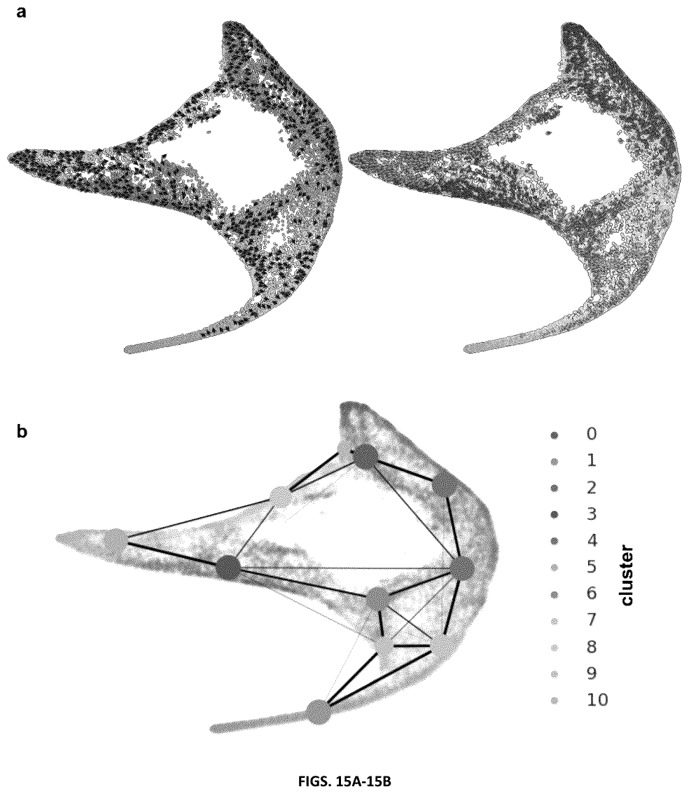

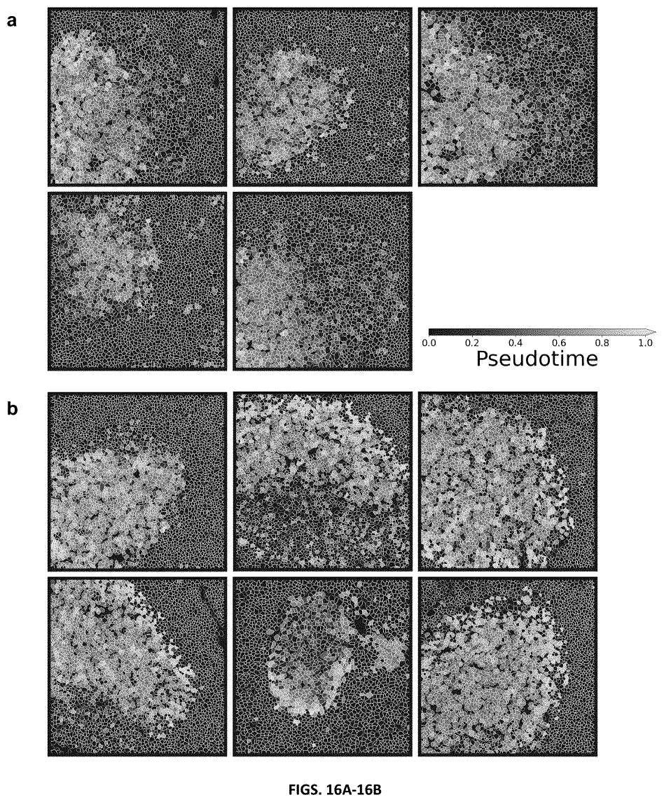

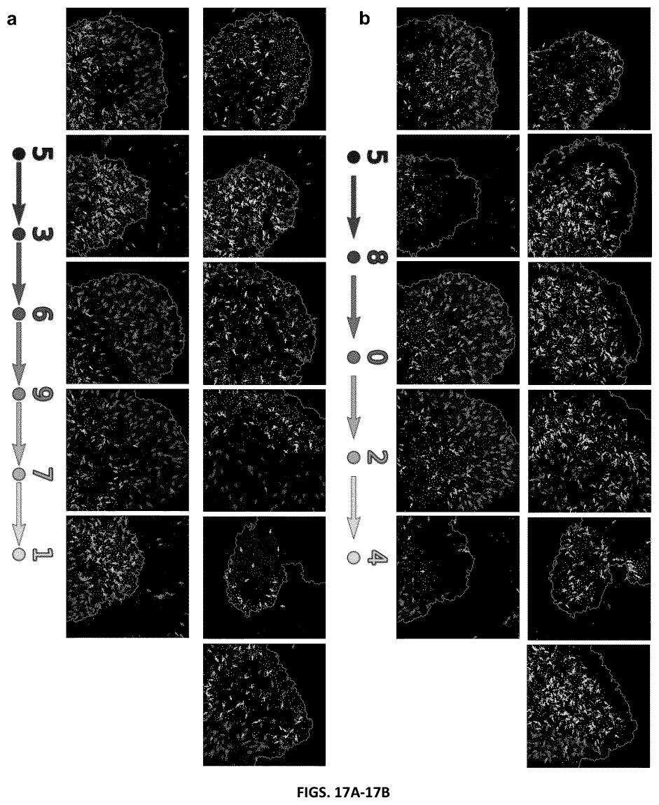

To visualize the B-cell trajectory gradient in the spatial domain, defined B-cell phenotypes were projected from trajectory analysis onto their spatial domain. Each cell was represented by a scatter point with the color corresponding to its cluster information. Following the identified trajectory paths for each cell in a cluster along a path, the spatial direction was defined by taking the five nearest neighbors of the cell in the next cluster on the path, and an arrow to the centroids of the nearest neighbors was plotted (FIG. 9D). This allows the spatial reconstruction of B-cell differentiation trajectory inside of the germinal center in human tonsil tissues. Similarly, the corresponding single-cell differentiation states were projected back into their TSNE embedding space for visualization and represented as a graph-directed method (FIGS. 15A-15B). On the other hand, the single-cell pseudotime values were projected into their spatial domain and the corresponding pseudotime trajectory gradient to visualize the spatial spread of these trajectories (FIGS. 16A-16B, FIGS. 17A-17B).

### Example 9—Spatial Metabolomics Profiling in Endometrium Tissues

Human endometrium, the mucous membrane lining the uterus, undergoes dynamic changes through remodeling, shedding, and regeneration during the menstrual cycle⁷². The temporal and spatial dynamics of endometrium cells have been studied at the single-cell level to dissect the signaling pathways that determine the cell fate of the epithelial lineages in the luminal and glandular microenvironments⁷³. Studies have shown that increased body mass index is most strongly associated with endometrial cancer incidence and mortality^{74,75}. It is therefore important to characterize the metabolomic variation between cell types and across conditions to better understand molecular mechanisms underlying how obesity contributes to endometrial cancer.

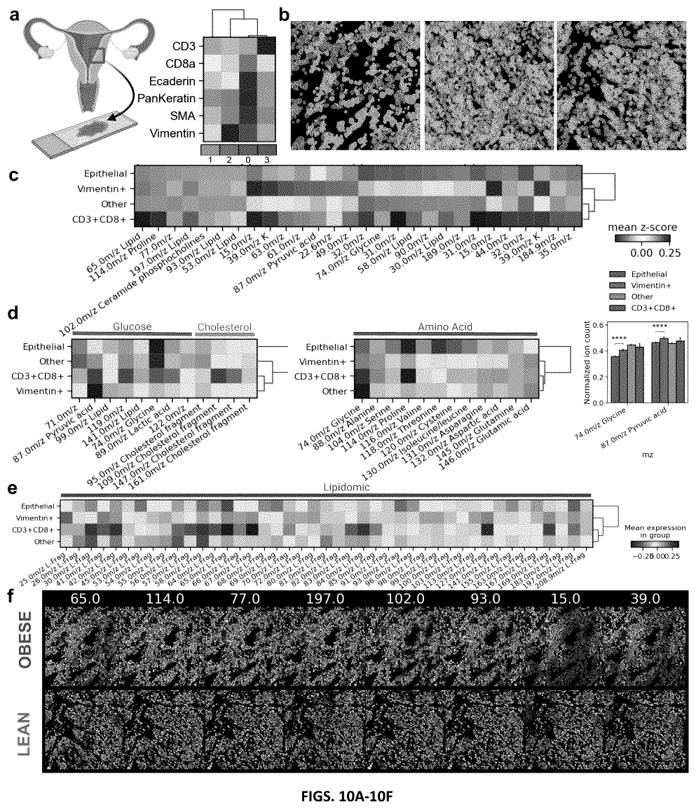

ScSpaMet was applied to human endometrium tissues to characterize their protein-metabolic environment. Herein, an antibody panel of 9 markers included immune surface markers, epithelial markers, and extracellular matrix proteins (TABLE 9). ScSpaMet first identified single-cell protein phenotypes (n=4) using the Leiden algorithm (FIG. 10A) and representative patients' tissue images were then reconstructed from the cell masks, and their clustering results by assigning each segmented cell to its corresponding cluster (FIG. 10B). In each patient tissue image, single-cell protein profiles were extracted (n=8125) to characterize cell phenotype, and a comparison of highly expressed metabolites for cell type was conducted (FIGS. 10D-10E). Single-cell metabolite spatial maps were reconstructed to visualize metabolite variation across regions in lean and obese patients' tissue (FIG. 10F). Next, the metabolomic variation between lean and obese patient samples was quantified by comparing the annotated mass channel associated with glucose, cholesterol, amino acids, and lipids. Glucose pathway-related channels such as 74.0 m/z Glycine and 89.0 m/z Lactic acids showed high expression in obese samples whereas other glucose fragments (71.0 m/z, 87.9 m/z Pyruvic acids, 99.0 m/z) showed higher expression in the lean patient.

TABLE 9
List of antibodies, their conjugated metal tags,
their clones, and their concentrations used for
imaging mass cytometry in endometrium tissues.

| Marker | Clone | Metal Tag | Dilution |
|---|---|---|---|
| CD3 | Polyclonal | 170Er | 1:100 |
| CD45RO | | 166Er | 1:50 |
| CD45RA | UCHL1 | 173Yb | 1:50 |
| CD8a | C8/144B | !62Dy | 1:100 |
| Pan-Keratin | C11 | 148Nd | 1:100 |
| SMA | 1A4 | 141Pr | 1:200 |
| E-Cadherin | 24E10 | 158Gd | 1:50 |
| Vimentin | D21H3 | 143Nd | 1:100 |
| Intercaltor | | 192/193Ir | 1:100 |

### Example 10—Spatial Metabolomics in Unstained Tissues

3D-SMF and IMC experiments were sequentially imaged after staining the cell with isotope-barcoded antibody libraries. This integrated protocol allows single-cell spatial metabolomics (scSpaMet) to detect both the nuclear and cytosolic distributions of metabolites and protein receptors in immune cells and cancer cells within the TME.

A spatially resolved three-dimensional (3D) metabolic profiling framework (3D-SMF⁷) is used to profile lipid depletion in germinal centers of human tissues. 3D-SMF has excellent potential to localize individual cell types by isotope-labeled antibodies against cell receptors and biomarkers, and profile metabolic maps of those localized cells in tissues in the same measurement. 3D-SMF utilizes a high-throughput and advanced spatial profiling method based on a time-of-flight secondary ion mass spectrometry (TOF-SIMS) instrument to image a 3D biopsy sample across 1-100 μm thick tissues. 3D-SMF integrated isotope-tagged antibodies to profile proteins in single-cells and MS to measure small molecules such as lipid distributions around these localized cell markers.

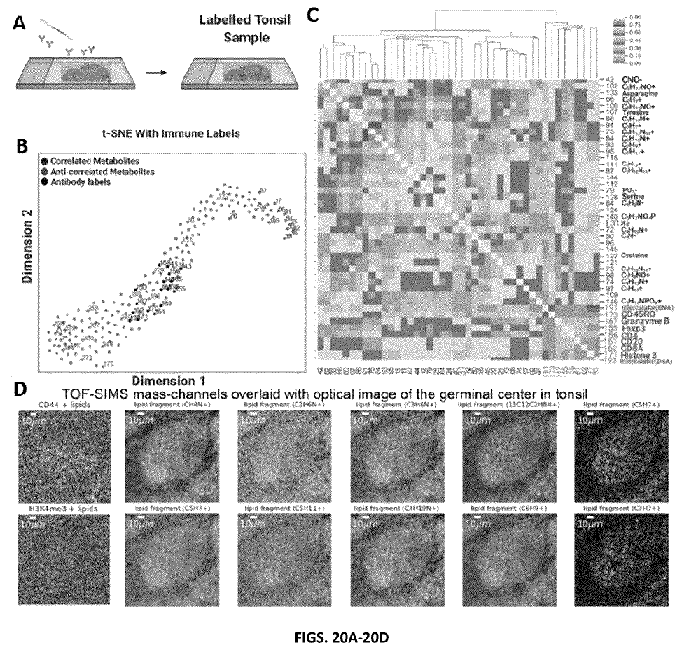

3D-SMF can generate 3D metabolite profiles and immune localization maps for 10-50 μm thick tissues, revealing the cell-to-cell interactions, extracellular matrix, and metabolic driving networks in lung tumors. The 3D-SMF method yielded unique lipid depletion and enrichments detected inside the germinal centers compared to outside (FIGS. 20A-20D). After fixing and permeabilizing the tissues, isotope-tagged antibodies were incubated with the tissue, followed by ethanol washes and drying the sample for 3D-SMF imaging.

3D-SMF was designed to enrich proteins by 5-fold in the MS readout, but the identification of cytosolic boundaries was challenging due to the inefficacy of secondary ion generation from limited isotope tags. IMC experiment followed 3D-SMF experiments to efficiently localize protein receptors in the same tissue.

The isotope-tagged antibody staining was performed before both 3D-SMF and IMC because the sample needs to be imaged in vacuum/dry conditions and high-quality labeling can only be done in wet samples using well-established isotope-antibody labeling protocols. After isotope tag staining, 3D-SMF imaged the top portion (1-2 μm thick) of cells and IMC imaged the rest of the cell thickness (3-4 μm thick), allowing to assign both metabolites and proteins to the same single-cell. IMC defines nuclear and cytosolic boundaries of single-cells at 1-μm precision and 3D-SMF provides direct correlations of proteins to the metabolites within IMC-defined cell segmentation masks. IMC's protein signal is different than that of 3D-SMF because they are imaging different portions of the same cell, providing indirect protein correlations to the metabolites of individual cells.

scSpaMet was developed in unstained and stained lung tumors using enhanced 3D-SMF and sensitive IMC method, followed by co-registration of image series and spatial bioinformatics analyses.

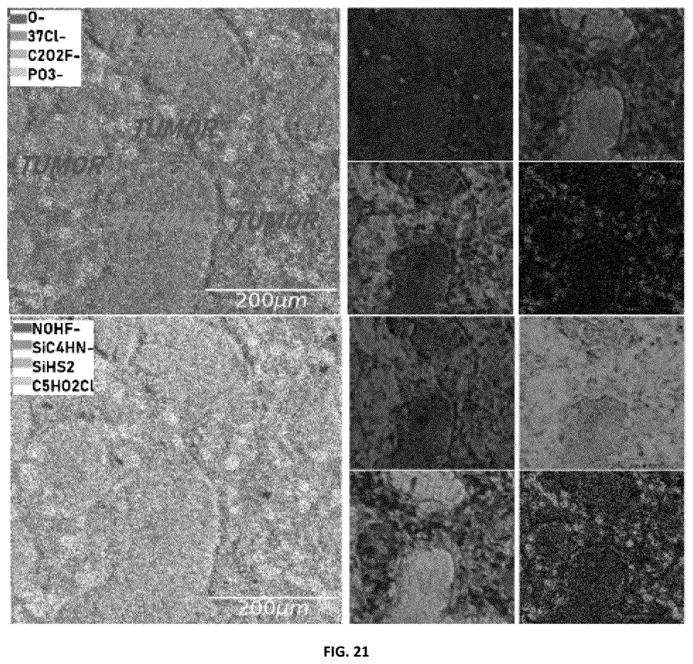

TOF-SIMS imaging was used to produce spatially resolved metabolic maps of an FFPE lung squamous cell carcinoma (Stage 1, grade 1, and T2N0M0: no metastasis, Biomax: LC10012a, A3) tissue for the 180 metabolic fragment channels. The composite images from multiple mass channels were overlaid (FIG. 21), demonstrating tumor regions with high PO3 (cell nuclear region) and stromal region (without cell content but extracellular matrix). This experiment showed that TOF-SIMS only lacked cytosolic boundaries of cells, but the nuclear regions were identified in phosphate rich channels (PO₃⁻ and PO₄). These nuclear features are used to register IMC images to the 3D-SMF images obtained by a TOF-SIMS experiment. Besides, these metabolic maps were only limited to detection of lipids and elements without cell-type information. Thus, unstained tissues serve as a control to the adjacent tissues that are stained by isotope libraries. This validation experiment is performed in an FFPE tissue microarray (BC04118a) from Biomax containing 3 serial sections each of Lung squamous cell carcinoma, including pathology grade, TNM and clinical stage, 50 cases/100 cores.

Data controls include >3 replicates of 3 adjacent tissue microarrays, >80% imaging reproducibility in experiments, SD and Mean (CV %) value for metabolic signal comparisons in tissues, >0.05 p-value in two-sided T-tests, >0.9 accuracies for pathology validation using Hematoxylin and Eosin (H & E) stain, >0.5 correlation between 30-markers in similar tissue types.

### Example 11—scSpaMet in Stained Tissues

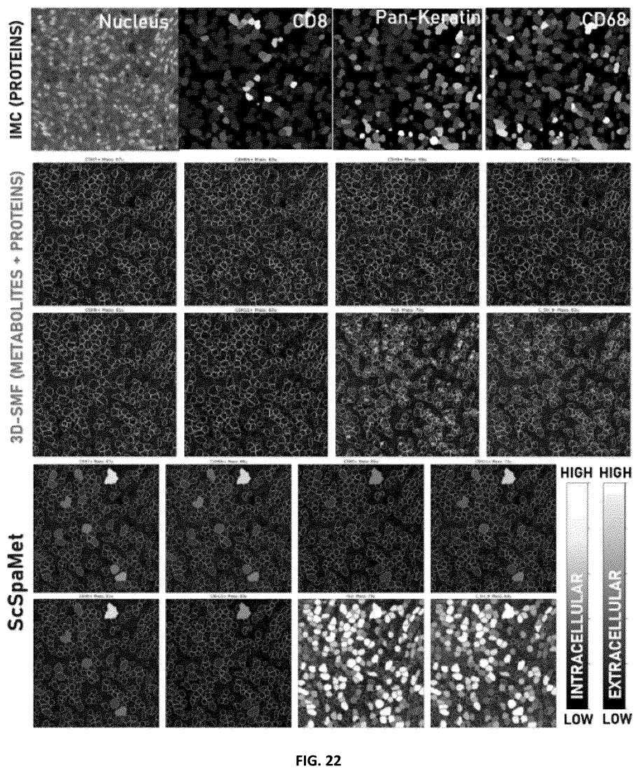

The 3D-SMF imaging was then performed in lung tissues (Biomax: BS04081a, triplicates per case). Several technical advancements were achieved to enhance specificity, sensitivity, and robustness. The experiment was demonstrated in A6 core corresponding to a lung adenocarcinoma tissue with grade 3, stage 2b, and T2bN1M0. First isotope labeling step included epithelial cells (148-Nd PanKeratin), T cells (170-Er CD3, 156-Gd CD4, 162-Dy CD8A, and 155-Gd Foxp3) and macrophages (159-Tb CD68), providing protein enrichments in 3D-SMF and IMC data. The preliminary data included lipids (e.g., C₅H₇) and elements (PO₃) detected in the 3D-SMF (black-orange colormap) and nucleus, CD8, pan-keratin, and CD68 positive cells identified in the IMC images (blue-red colormap), providing single-cell distributions of metabolites (FIG. 22). After segmenting the IMC images using Cellpose²⁸ deep learning algorithm, single-cells were identified and cell boundaries were colored by white (pan-keratin, cancer cell), green (CD8 T cell), and magenta (Cd68 cells). 3D-SMF images were also extracted for lipids and elements (Orange). These two datasets were combined using nucleus (IMC) and PO3 (3D-SMF) to register two multiplexed metabolic and protein imaging datasets. The alignment was performed by a cross-correlation algorithm to correct rotational and translational shifts in images at 300-nm pixel accuracy. scSpaMet maps then included two colormaps: black to orange for metabolites inside the cell (intracellular) and black to gray for metabolites outside the cell (extracellular). Cell types were also marked by white, green, and magenta boundaries to associate metabolites to cancer cells, CD8 cells, and CD68 cells, respectively. Thus, scSpaMet maps in-situ metabolic profiles of single-cells with 10-fold higher resolution than SpaceM.

Next, the scSpaMet pipeline was performed in the same lung microarray (BS04081a) containing 63 cores and 3 replicates for each patient. The adjacent tissue section was left unstained and compared to the stained sections to demonstrate the efficacy of staining and 90% threshold in the quantification was used to distinguish any background from glass specimen and other non-specific signals from the tissues.

Statistical >95% confidence using ANOVA with Bonferroni or Tukey's test was used to compare spatial metabolic profiles of 63 tissues from 21 patients. Antibodies used for proteins were benchmarked by immunohistochemistry to show 80% specificity. The dynamic range of metabolic signal was adjusted to 90% across multiple tissues and normalized by z-score analysis. SD & Mean (CV %) values were used for subcellular expression and localization comparisons of metabolites across three replicates of the same tissue.

### Example 12—Optimization of scSpaMet in Stained Tissues

The 3D-SMF was enhanced through these steps:

Selection of isotopes for enhancing signal of each antibody target: Immune cells were localized by 3D-SMF in lung tumors using isotope-tagged CD8, CD4, CD68, Foxp3, CD3, and CD45 markers. Each marker was stained using immunohistochemistry in single lung tissue sections and signal levels of each marker was ranked from low to high expression. Pure isotope targets were characterized using lanthanides (¹⁴¹Pr, ¹⁴³⁻¹⁴⁸⁻¹⁵⁰Nd, ¹⁴⁹Sm, ¹⁵⁵⁻¹⁵⁸Gd, ¹⁵⁹Tb, ¹⁶¹⁻¹⁶²Dy, ¹⁶⁵Ho, ¹⁶⁷⁻¹⁶⁸⁻¹⁷⁰Er, ¹⁶⁹Tm, ¹⁷¹⁻¹⁷³Yb, ¹⁷⁵Lu, and ¹⁹¹⁻¹⁹³Ir) and another set of halogens (²H, ¹⁹F, ^{79/81}Br, and ¹²⁷I) to determine sensitivity of each isotope molecule detected by the TOF-SIMS machine. Isotopes were assigned to the antibody for obtaining the higher scSpaMet sensitivity (>20-50%) same lung microarray (BS04081a) containing 63 cores and 3 replicates for each patient.

Selection of ion source for increasing secondary ions: A different ion source was tested using an Oxygen gun to enhance isotope detection sensitivity in TOF-SIMS data. The same lung tissue was imaged with Oxygen or Bismuth sources (Bi⁺ and Bi³⁺) to compare efficacy of secondary ion extraction per isotope. Pure targets from the previous optimization determined the isotopes converted to secondary ions (% ion conversion efficiency=isotope density divided by number of secondary ions), followed by re-assignment of isotopes to biomarkers for distinct set of experiments using oxygen or Bismuth source in BS04081a microarray. Ion source optimizations boosted the sensitivity by 5-fold via enhancement of ion efficiency from 5% to 25%.

Characterization of metabolite and antigen quality by tissue preservation: scSpaMet was tested in 5-μm thick Formalin-fixed and paraffin-embedded (FFPE) and 5-μm thick fresh frozen lung tissues to determine the antigen quality and metabolite retention for immune cells. A fresh tissue block (AMSBIO, 100028001) was sliced into pieces with 1-mm each, half of which was fixed and the other half was kept frozen. The tissue sections from these paired samples provided the metabolite and protein concentrations, providing the effect of fixation. This experiment provided less than 3-fold metabolite and protein loss as a standard of scSpaMet.

### Example 13—scSpaMet in Native Tissues

Metabolism of immune cells in tumors has played a vital role in understanding functional change in immune and cancer cell interactions.²⁹ Cancer cells rapidly proliferate and deplete nutrients in the tumors, making it challenging for immune and stromal cells to survive. Each cell type alters its metabolism to properly function using rearrangement of metabolic pathways to regulate fatty acids, lipids, amino acids, glucose, oxygen, and energy, among many others.³⁰ Lymphocytes rewire their intracellular metabolic states to overcome cancer-induced barriers in TME, allowing them to infiltrate cancer regions efficiently.³ A unique metabolic program regulated by lipids and fatty-acid-oxidation (FOA) events guides immune infiltration, causing hot or cold tumors are tested; and these programs are different in histologic subtypes of lung cancers. scSpaMet localizes the immune infiltration patterns of lymphocytes (CD4, CD8, and CD3) and myeloid cells (CD68) using the isotope-tagged antibody libraries identified in the imaging mass-spectra. Metabolic lipid distributions around these cells were then measured for about 160 metabolites with hundred lipids using the same 3D-SMF method in lung cancer tissues to study the lipidomic mechanisms in lung cancer biology.

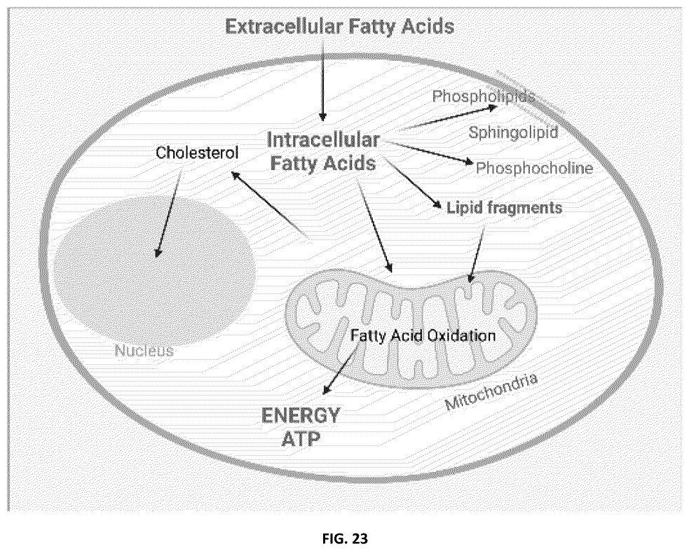

Lipid metabolism was upregulated in lung cancers.³¹ A few significant metabolic genes involved in fatty acid metabolism were associated with lung cancer prognosis. Furthermore, cancers' basic biology showed direct relation of lipid metabolism and cell migration, invasion, and angiogenesis found in tumors.³² FAO is a catabolic pathway that breaks down the fatty acids into cetyl-CoA, NADH, and FADH2 to produce energy, but Fatty acid synthesis (FAS) converts acetyl-CoA into lipids in the cytosol (FIG. 23). FAS enzymes were upregulated in lung cancers to increase FAO in mitochondria for producing energy while depleting Fatty acids (FAs).³³ Besides, FAS supports proliferation of T-cells and emerging drugs can target FAS mechanisms to inhibit tumor growth in NSCLCs.³⁴ The multiplayer role of metabolism includes tumor-associated macrophages (TAMs) contributing to cancer cell and lymphocytes' interactions.³⁵ Recent immunotherapy designs showed essential metabolic genes and pathways to predict response and boost immune-based therapies in lung cancers.^{36,37} This growing body of evidence indicates the need for a highly multiplex assay to identify immune cell subtypes, their locations, and metabolism in the TME to improve outcomes. High-throughput scSpaMet profiles tissue microarray (Biomax: LC953) from distinct lung cancer patients with adenocarcinoma (n=30) and squamous cell carcinoma (n=30). Ten of these tissues for both histologic subtypes are also compared to matched metastatic lymph nodes, providing the role of metabolism in metastasis. Spatial single-cell imaging creates a tumor map for potentially guiding lung disease treatments.

### Example 14—scSpaMet in Tissues from Lung Adenocarcinoma

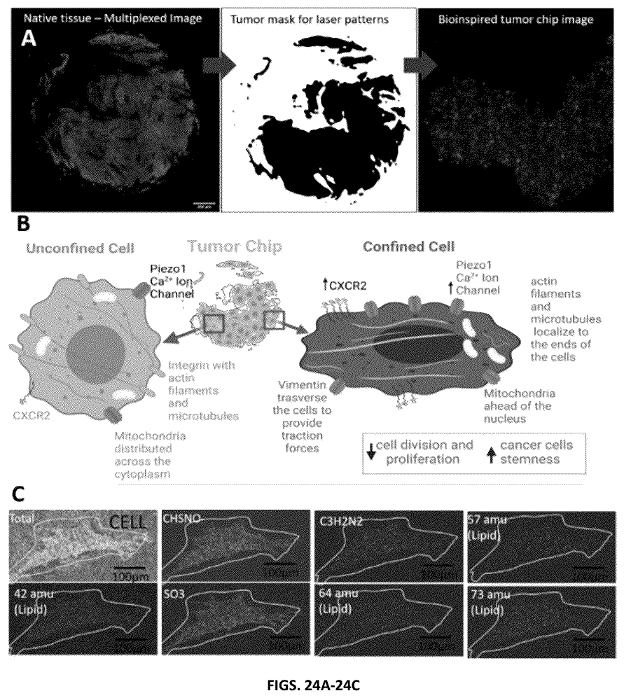

FAO regulates immune infiltration, leading to cold tumors (FIGS. 24A-24C).³⁸ This was tested by profiling FAs in cancer cells in hot and cold tumors using the localized immune cell patterns in the most abundant lung cancers, adenocarcinomas. ScSpaMet targeted 20 isotope-conjugated markers [epithelial cells (148-PanKeratin), T-cells (170-CD3, 156-CD4, 162-CD8A, 155-Foxp3, and 173-CD45RO) Myeloid cells (159-CD68), nucleus (171-Histone 3 and 191/193-Intercalators), and functional markers (143-Vimentin, 158-E-Cadherin, 168-Ki67, and 167-Granzyme B)] for identifying cell types and up to 150 metabolites in lung adenocarcinoma (n=50) cancer microarrays (Biomax: LC10013c, Tissue core size=1 mm×1 mm), providing lipid depletion or enhancement in immune and cancer cells within distinct parts of the tissues. Metabolic profiles of these localized cells focused on lipid metabolism to profile FAs (C₃H₁₀N⁺, C₅H₁₄N⁺, among others⁷) and cholesterol (C₂₃H₄₅O₄⁻). scSpaMet mapped out the subset of FAs in cancer cells in spatial relation to the subgroup of infiltrating immune cells. For instance, myeloid cells' immune suppression due to FAO were studied compared to lymphocytes experiencing less suppression, providing correlative insights into CD8A infiltrates more than CD68 macrophages. Matched adjacent normal tissues (n=50) were compared to the tumor sites for differential analysis of metabolic and lipid changes in cancer regions. Additional twenty metastatic lymph node sections (Biomax: LC814a & LC817b) matching to the primary tumors were measured to compare metabolic and lipid profiles between the primary tissues and metastatic sites.

Data controls included >3-replicates of tissue microarrays, >80% staining reproducibility in experiments, SD and Mean (CV %) value for tissue comparisons, >0.05 p-value in two-sided T-tests, >0.9 accuracies for pathology validation, >0.5 correlation between 30-markers in similar tissue types. FAO levels were benchmarked by independent fluorometric assays (Abcam, ab222944) and MALDI imaging to validate high-low FAO.

Discussion: Immune cell types experience significant metabolic programming when they infiltrate the tumor ecosystem.² Cancer progression controls multiple immune and stromal cell types and their metabolic functional roles in tumors due to the rapid nutrient depletion and accumulation of waste products during rapid proliferation.³ Gene expression profiling, protein analysis, and metabolic mapping have been used to study the metabolism of the tumor microenvironment (TME).⁴⁻⁶ Currently, these molecular assays are typically performed in separate clinical steps with incompatible technical variations. Thus, an integrated measurement technique is needed to decipher the TME's complexity covering multiple immune cell types, spatial immune infiltration patterns, and several metabolic pathway alterations.

Inflammation balances tissue homeostasis in injuries and infection in the human body.¹⁹ The immune system and metabolic pathways coordinate with each other to regulate the inflammatory response.²⁰ Many diseases, including cancers, diabetes, neuronal disorders, utilize this complex interplay of immune cells and metabolic regulation.^{2,21,22} Infiltration of immune cells in tissues indicated dysfunctionality of local solid tumors, and this process results in a metabolic imbalance in tumors and other tissues. Thus, it is essential to study the intricate balance of metabolism and immune infiltration using molecular analysis methods to shed light on tissue biology. This project studies the TME to decipher the intersection of immunity and metabolism in lung cancer biology.

Lung cancer remains to be one of the most leading causes of death worldwide.²³ Cancer immunotherapy approaches have revolutionized lung cancer treatment using immune checkpoint inhibitors. However, these immunotherapies are only efficient for a small subset of lung cancer patients.²⁴ In patients treated with checkpoint inhibitions, differential spatial and temporal responses were observed based on the immune-rich or immune-cold tumor lesions.²⁵ The main drivers of treatment response variability in distinct patients are the heterogeneous molecular and cellular compositions of the tumors and immune infiltrate between lung patients.²⁶ Thus, it is crucial to quantify infiltrating immune in lung cancers using spatially resolved metabolic imaging techniques for mapping each cell's locations with molecular data. Multiplex imaging technologies develop a comprehensive understanding of the immune organization in native tissue at the single-cell level resolution to address these issues associated with the previous imaging methods. This multiplexed molecular profiling method provides metabolic and immune localization maps in tumors.

### Example 15—scSpaMet in Tissues from Squamous Cell Carcinomas

Histological subtypes exhibit different metabolic regulation in tumors. Adenocarcinomas and squamous cell carcinomas show differences in lipid and fatty acid oxidation-based metabolic regulation.³⁹ A study compared 3D metabolic profiles and immune localization patterns obtained from scSpaMet to another lung cancer subtype known as squamous cell carcinomas (SqCC). The critical question here was to relate lipid metabolism and immune infiltration (hot vs. cold) in distinct patient cohorts using a microarray specimen (Biomax, BC04118a) that contains SqCCs (n=50) including the primary sites and matched adjacent normal tissues (n=50). These scSpaMet experiments shed light on the interplay between metabolism and immune infiltration in distinct lung cancer subtypes. Differential metabolic expression maps were created from both histologic subtypes.

The analytical tools⁷ were repurposed to analyze 3D metabolic networks of lung tissues from adenocarcinomas and SqCCs. Statistical significance analysis of p-value <0.05 (Wilcox, two-sided) was also used to compare markers and metabolites' intensities in both cancer types. Image cytometry analysis on 3D-SMF gate immune and other cell phenotypes from isotope-tagged mass-data. Metabolic peaks were systematically identified and validated using mass-number predictions. A “spatial score” was created to associate immune infiltration in hot and cold tumors, which can then be plotted using differential enrichment analysis from 50 adenocarcinomas and 50 SqCC tissues. For clustering analysis, F-1 score calculations were performed to study spatial similarities.

In general, NSCLCs occur both in males and females; a balanced mix was used in tissue studies. Age spanned a range of 35-75 with an average closer to older populations as the NSCLCs are more prevalent in the aged population.

### Example 16—Linking Inflammation to the scSpaMet Maps

A subset of immune cell types infiltrates cancer regions to initiate the elimination of cancers. However, other immune cells, such as granulocytes and Tregs, also play a role in suppressing tumor immunity. Thus, the spatial patterns⁴⁰ of immune infiltrated tumors can be summarized as follows.

Hot tumors (complete): Patients exhibit high T-cell infiltration by CD8, CD3, and CD4 cells.^{41,42}

Cold tumors (immune-ignored): Patients may exhibit almost no CD8 CTLs but rather other cells inhibiting T-cell activity against tumors.

Cold excluded tumors: Patients show immune infiltration at the peripheral region of a TME, but these immune cells fail to infiltrate the core of TMEs.

Cold tumors (immune-desert): Patients demonstrate no existence of immune cells in the TMEs.

scSpaMet localizes major immune cell types to categorize lung tumors into one of these four major spatial patterns. A study sought to determine the molecular drivers of these unique spatial patterns using the scSpaMet's metabolic profiles and correlation to the localized immune and cancer cells in fifty tissues in the microarrays profiled in all the lung tissues (LC10013c, BC04118a, LC814a, LC817b, & LC953). Extracellular metabolites created gradients to direct cells to spatial regions of tumors. scSpaMet then correlated spatially resolved metabolomics maps to localize lymphocytes and myeloid cells for studying the tumor phenotypes used in the hot and cold classifications in lung adenocarcinomas and sqCCs. Cancer stages, tumors, metastasis, and survival from these tumor tissues were classified, potentially correlated with lung disease progression as a function of metabolic profiles and infiltrates.

Survival was estimated using the Kaplan-Meier method and the impact of metabolism, immune profile, and SABV on survival was evaluated using Cox proportional hazards models. Metabolic differences can be detected with 80% power and 0.05 significance level using more than hundred tissue cores for each histologic subtype. All experiments were done in triplicate.

### Example 17—scSpaMet in Bioinspired Tumor Chips

MSI is an emerging field that captures 4D metabolite information (i.e., localization and chemical information) to address the loss of spatial details in the bulk level MS techniques.¹² Several methods have allowed MSI capability, including MALDI, desorption electrospray ionization (DESI), and secondary ion mass spectrometry (SIMS).¹³ These methods and their derivatives utilize ionization methods exposed to the samples of interest (e.g., hard or soft materials) to produce gas-phase ions, followed by an analysis of mass to charge ratio (m/z) detected by a mass spectral analyzer. Typically, biological samples (e.g., cells and tissues) are at the core of this proposal and they are chemically fixed and treated with a sample preparation method (e.g., matrix deposition) and ionized by an electrospray, a laser beam, or an ion beam. The resultant gas ions are then detected by an ion trap mass spectrometer and time-of-flight (TOF) mass spectrometer, revealing the identity of metabolites, lipids, and proteins, along with their spatial coordinates.¹⁴ MALDI and DESI efficiently map metabolites at 20-100 μm spatial resolution and TOF-SIMS acquires lipids at sub-micron spatial details. MALDI and DESI have analyzed three-dimensional (3D) volumes of biological tissues from model organisms (e.g., mouse and zebrafish) using serial tissue sections, followed by digital assembly of 3D metabolic profiles.^{15,16} On the other hand, TOF-SIMS and OrbiSIMS have mapped 3D tissue compositions of tissues by sequentially ablating and imaging thin layers, providing 3D metabolic reconstructions.^{17,18} These results have obtained snapshots from samples fixed or frozen at a single time point, but real-time MSI has also been implemented by iKnife in vivo at low spatial resolution. Therefore, spatial metabolomics is feasible in 4D using MSI technologies and digital analysis of resultant 3D image data cubes across diverse samples.

Provided herein is a bio-inspired tumor-on-a-chip system using the spatial form factors obtained from the multiplexed imaging data. Rationale is to mimic tumor boundaries that can regulate immune cell infiltration based on differential metabolic gradients. Thus, microfluidic patterning masks are generated from the patients' multiplexed tissue images that show the spatial organization of cancer cells and immune cells. This organization can be recapitulated using in vitro microfluidic chips to develop on-chip tumor models of lung cancer. Proof-of-principle was demonstrated from a cytokeratin stained tumor sample (purple) and the mask is generated from this native tissue image using laser cutters of a 0.25 mm thick Polydimethylsiloxane (PDMS) film (FIG. 24A). The PDMS film was then bound to a coverslip and 0.5-2 million A549 lung cancer cells (established lung adenocarcinoma model) for 2-days, followed by fixation and single-cell metabolic analysis. In this tumor-chip, cells closer to the PDMS membrane experience “confinement” that modifies their subcellular structure and function. For instance, organelles are asymmetrically distributed, calcium flux is altered, cell division is reduced, and structural actins are polarized in cancer cells (FIG. 24B)⁴³⁻⁴⁵. scSpaMet analysis of these confined cells from the tumor-chip shows the effect of tumor boundaries and how they create metabolic gradients to guide cell decisions, akin to single-cell metabolic images (FIG. 24C).

The bioinspired tumor-on-a-chip model exhibit unique benefits:

Modular and open microfluidics: Custom tumor shapes can be patterned for studying confinement configurations. Assembly and removal of PDMS is also flexible, allowing physical access to the cells for molecular profiling. Spatial formats of 1:3, 1:5, 1:10, and 1:20 ratios are used to design PDMS patterns and cell densities are varied from 0.1-5 million/mL cells to quantify the effect of cell density on metabolism.

Spatio-temporal multi-cell dynamics: Multicellular cultures are feasible. After culturing cancer cells for 1-7 days and CD8 cells ATCC, PCS-800-017) are loaded to the open-microfluidics from a vessel-like thin channel to mimic infiltration-on-a-chip. CD8-A549 cell neighbors are analyzed by scSpaMet.

At least five replicates for each condition. >30% survival for cells and >50% cell viability for CD8+ T-cells. >1, 3, 5, and 7-days cultures are used for cell viability. <0.05 p-value is significant in cell counts. >80% cell confluency is used for co-cultures of A549 with T-cells. >1,000 cells per well are analyzed.

Discussion: 3D-SMF maps lipids with high confidence, but several metabolites, including glucose and glycolysis pathways, may not be captured efficiently due to inefficiency during the secondary ion generation. Thus, enzymes encoding for these pathways are tagged using antibodies and isotope barcodes, similar to another T-cell metabolic analysis work.⁴⁶ The system disclosed herein allows the detection of multiple metabolic pathways that coordinate immune and cancer cell function with lipid metabolism. Extracellular metabolites may not have been captured accurately during the tissue fixation, but abundant metabolites should still have some residues for spatial tissue mapping. The upregulation or depletion of lipids in tumors may be weak in some metabolic channels. Adjacent normal tissue (AT) is used to determine the differential change in tumors. Besides, tumor's cell complexity would have been partially captured in metabolic analysis. Thus, a subset of T-cell lymphocytes⁴⁷, quiescent and active T-cells in the tumors^{48,49}, and APCs such as dendritic cells (CD11c+MHC-II+) or macrophages (CD68+CD11b+) are targeted to study the tumor's metabolism in local niches with T-cell subtypes, APC enrichments, and their relative spatial patterning in the microenvironment.

The presented high-risk platform develops a single cell spatial metabolomics platform to profile lipids, elements, and protein signatures in a single platform within complex tissues. Single cell spatial metabolomics integrates untargeted metabolite imaging and targeted protein mapping in the same lung tissues and bioinspired tumor chips. scSpaMet in native tissues provides the snapshots of metabolic regulation in patients' tissues, while it generates biodynamics of metabolic regulation guiding the immune and cancer interactions within physical confinements in bioinspired tumor chips. This emerging technology impacts understanding of the complex FAO, FAS, and lipidomic dynamics of cancer and immune cells, providing a mechanistic understanding of why some patients demonstrate more immune infiltration in the tumors. Spatially regulated metabolic control finds use in the treatment of life-threatening diseases such as cancers and beyond.

The key role of metabolic regulation in a cell and tissue-specific context is recently emerging as a new paradigm at the intersection of cancer biology and immunology. This system addresses the complexity of immune infiltration in tumors at the single-cell and subcellular level by testing whether lipid distributions can be used to pinpoint unique immune cell functions related to cancer progression and elimination.

Technically, existing metabolic imaging technologies remain insufficient for single-cell mapping of this metabolic heterogeneity. This system presents image-based single-cell spatial metabolomic profiling by integrating untargeted metabolomics and targeted protein profiling in a single platform to validate the metabolic significance of the specific cellular decision making. This untapped region of the single-cell spatial metabolomics opens a new area of investigation by systematic high-throughput metabolic mapping of multicellular communications.

The heterogeneity of this metabolic and lipid profiling system in a complex tissue is explored down to a single-molecule and single-cell level at a high-throughput screening of 200 metabolites and 35 proteins. A cell's boundary is localized through identification of nucleus, cell membrane, and extracellular matrix in scSpaMet data. Single-cell metabolic control uncovers the mechanisms of divergence from lipidomics homeostasis through the dysregulation of oxidation and energy generating factors in native biopsies and bioinspired tumor-on-chips.

The following patents, applications and publications as listed below and throughout this document are hereby incorporated by reference in their entirety herein.

### REFERENCE LIST FOR EXAMPLES 1-9

1. Wellen, K. E. & Thompson, C. B. A two-way street: reciprocal regulation of metabolism and signalling. Nat. Rev. Mol. Cell Biol. 13, 270-276 (2012).
2. Kim, J. & DeBerardinis, R. J. Mechanisms and Implications of Metabolic Heterogeneity in Cancer. Cell Metab. 30, 434-446 (2019).
3. Leone, R. D. & Powell, J. D. Metabolism of immune cells in cancer. Nat. Rev. Cancer 20, 516-531 (2020).
4. Biswas, S. K. Metabolic Reprogramming of Immune Cells in Cancer Progression. Immunity 43, 435-449 (2015).
5. Renner, K. et al. Metabolic Hallmarks of Tumor and Immune Cells in the Tumor Microenvironment. Front. Immunol. 8, (2017).
6. Andrejeva, G. & Rathmell, J. C. Similarities and Distinctions of Cancer and Immune Metabolism in Inflammation and Tumors. Cell Metab. 26, 49-70 (2017).
7. Lim, A. R., Rathmell, W. K. & Rathmell, J. C. The tumor microenvironment as a metabolic barrier to effector T cells and immunotherapy. eLife 9, e55185 (2020).
8. Buchberger, A. R., DeLaney, K., Johnson, J. & Li, L. Mass Spectrometry Imaging: A Review of Emerging Advancements and Future Insights. Anal. Chem. 90, 240-265 (2018).
9. Vickerman, J. C. Molecular imaging and depth profiling by mass spectrometry-SIMS, MALDI or DESI? The Analyst 136, 2199 (2011).
10. Kompauer, M., Heiles, S. & Spengler, B. Atmospheric pressure MALDI mass spectrometry imaging of tissues and cells at 1.4-μm lateral resolution. Nat. Methods 14, 90-96 (2017).
11. Sun, C. et al. Spatially resolved metabolomics to discover tumor-associated metabolic alterations. Proc. Natl. Acad. Sci. 116, 52-57 (2019).
12. Sjövall, P., Lausmaa, J. & Johansson, B. Mass Spectrometric Imaging of Lipids in Brain Tissue. Anal. Chem. 76, 4271-4278 (2004).
13. Rappez, L. et al. SpaceM reveals metabolic states of single cells. Nat. Methods 18, 799-805 (2021).
14. Yuan, Z. et al. SEAM is a spatial single nuclear metabolomics method for dissecting tissue microenvironment. Nat. Methods 18, 1223-1232 (2021).
15. Giesen, C. et al. Highly multiplexed imaging of tumor tissues with subcellular resolution by mass cytometry. Nat. Methods 11, 417-422 (2014).
16. Jackson, H. W. et al. The single-cell pathology landscape of breast cancer. Nature 578, 615-620 (2020).
17. Bodenmiller, B. Multiplexed Epitope-Based Tissue Imaging for Discovery and Healthcare Applications. Cell Syst. 2, 225-238 (2016).
18. Ganesh, S. et al. Spatially resolved 3D metabolomic profiling in tissues. Sci. Adv. 7, (2021).
19. Haghverdi, L., Lun, A. T. L., Morgan, M. D. & Marioni, J. C. Batch effects in single-cell RNA-sequencing data are corrected by matching mutual nearest neighbors. Nat. Biotechnol. 36, 421-427 (2018).
20. Hie, B., Bryson, B. & Berger, B. Efficient integration of heterogeneous single-cell transcriptomes using Scanorama. Nat. Biotechnol. 37, 685-691 (2019).
21. Barkas, N. et al. Joint analysis of heterogeneous single-cell RNA-seq dataset collections. Nat. Methods 16, 695-698 (2019).
22. Zhu, B. et al. Robust single-cell matching and multimodal analysis using shared and distinct features. Nat. Methods (2023) doi:10.1038/s41592-022-01709-7.
23. Hao, Y. et al. Integrated analysis of multimodal single-cell data. Cell 184, 3573-3587.e29 (2021).
24. Liu, J. et al. Jointly defining cell types from multiple single-cell datasets using LIGER. Nat. Protoc. 15, 3632-3662 (2020).
25. Seninge, L., Anastopoulos, I., Ding, H. & Stuart, J. VEGA is an interpretable generative model for inferring biological network activity in single-cell transcriptomics. Nat. Commun. 12, 5684 (2021).
26. Tran, D. et al. Fast and precise single-cell data analysis using a hierarchical autoencoder. Nat. Commun. 12, 1029 (2021).
27. Minoura, K., Abe, K., Nam, H., Nishikawa, H. & Shimamura, T. A mixture-of-experts deep generative model for integrated analysis of single-cell multiomics data. Cell Rep. Methods 1, 100071 (2021).
28. Anchang, B. et al. Visualization and cellular hierarchy inference of single-cell data using SPADE. Nat. Protoc. 11, 1264-1279 (2016).
29. Haghverdi, L., Buttner, M., Wolf, F. A., Buettner, F. & Theis, F. J. Diffusion pseudotime robustly reconstructs lineage branching. Nat. Methods 13, 845-848 (2016).
30. Palla, G. et al. Squidpy: a scalable framework for spatial omics analysis. Nat. Methods 19, 171-178 (2022).
31. Martinelli, A. L. & Rapsomaniki, M. A. ATHENA: analysis of tumor heterogeneity from spatial omics measurements. Bioinformatics 38, 3151-3153 (2022).
32. Pechuan-Jorge, X. et al. SPEX: A modular end-to-end analytics tool for spatially resolved omics of tissues. http://biorxiv.org/lookup/doi/10.1101/2022.08.22.504841 (2022) doi:10.1101/2022.08.22.504841.
33. Gradeci, D., Bove, A., Charras, G., Lowe, A. R. & Banerjee, S. Single-cell approaches to cell competition: High-throughput imaging, machine learning and simulations. Semin. Cancer Biol. 63, 60-68 (2020).
34. van Neerven, S. M. & Vermeulen, L. Cell competition in development, homeostasis and cancer. Nat. Rev. Mol. Cell Biol. (2022) doi:10.1038/s41580-022-00538-y.
35. Hu, T., Coskun, A. & Mayar Allam. Spatially visualized single-cell pathology of highly multiplexed protein profiles in health and disease. (2021) doi:10.5281/ZENODO.4662853.
36. Allam, M. et al. Spatially variant immune infiltration scoring in human cancer tissues. Npj Precis. Oncol. 6, 60 (2022).
37. Traag, V. A., Waltman, L. & van Eck, N. J. From Louvain to Leiden: guaranteeing well-connected communities. Sci. Rep. 9, 5233 (2019).
38. Wolf, F. A., Angerer, P. & Theis, F. J. SCANPY: large-scale single-cell gene expression data analysis. Genome Biol. 19, 15 (2018).
39. Schapiro, D. et al. histoCAT: analysis of cell phenotypes and interactions in multiplex image cytometry data. Nat. Methods 14, 873-876 (2017).
40. Almet, A. A., Cang, Z., Jin, S. & Nie, Q. The landscape of cell-cell communication through single-cell transcriptomics. Curr. Opin. Syst. Biol. 26, 12-23 (2021).
41. Kingma, D. P. & Welling, M. Auto-Encoding Variational Bayes. Preprint at http://arxiv.org/abs/1312.6114 (2014).
42. Saelens, W., Cannoodt, R., Todorov, H. & Saeys, Y. A comparison of single-cell trajectory inference methods. Nat. Biotechnol. 37, 547-554 (2019).
43. Melsen, J. E., van Ostaijen-ten Dam, M. M., Lankester, A. C., Schilham, M. W. & van den Akker, E. B. A Comprehensive Workflow for Applying Single-Cell Clustering and Pseudotime Analysis to Flow Cytometry Data. J. Immunol. 205, 864-871 (2020).
44. Giacomini, I. et al. Cholesterol Metabolic Reprogramming in Cancer and Its Pharmacological Modulation as Therapeutic Strategy. Front. Oncol. 11, 682911 (2021).
45. Gupta, S., Roy, A. & Dwarakanath, B. S. Metabolic Cooperation and Competition in the Tumor Microenvironment: Implications for Therapy. Front. Oncol. 7, (2017).
46. Yang, D., Guo, P., He, T. & Powell, C. A. Role of endothelial cells in tumor microenvironment. Clin. Transl. Med. 11, (2021).
47. Yeh, W. L., Lin, C. J. & Fu, W. M. Enhancement of Glucose Transporter Expression of Brain Endothelial Cells by Vascular Endothelial Growth Factor Derived from Glioma Exposed to Hypoxia. Mol. Pharmacol. 73, 170-177 (2008).
48. Wu, F. et al. Single-cell profiling of tumor heterogeneity and the microenvironment in advanced non-small cell lung cancer. Nat. Commun. 12, 2540 (2021).
49. de Sousa, V. M. L. & Carvalho, L. Heterogeneity in Lung Cancer. Pathobiology 85, 96-107 (2018).
50. Brandtzaeg, P. The B-cell development in tonsillar lymphoid follicles. Acta Oto-Laryngol. Suppl. 523, 55-59 (1996).
51. Lettau, M., Wiedemann, A., Schrezenmeier, E. V., Giesecke-Thiel, C. & Dörner, T. Human CD27+ memory B cells colonize a superficial follicular zone in the palatine tonsils with similarities to the spleen. A multicolor immunofluorescence study of lymphoid tissue. PLOS ONE 15, e0229778 (2020).
52. Choi, S. C. & Morel, L. Immune metabolism regulation of the germinal center response. Exp. Mol. Med. 52, 348-355 (2020).
53. Weisel, F. J. et al. Germinal center B cells selectively oxidize fatty acids for energy while conducting minimal glycolysis. Nat. Immunol. 21, 331-342 (2020).
54. Jiang, S. Dietary Fat Makes Germinal Center B Cells Happy. Cell Metab. 31, 890-891 (2020).
55. Pae, J. & Victora, G. D. B is for ‘Big Mac’: GCs crave a high-fat diet. Nat. Immunol. 21, 249-250 (2020).
56. Stebegg, M. et al. Regulation of the Germinal Center Response. Front. Immunol. 9, 2469 (2018).
57. Akkaya, M. & Pierce, S. K. From zero to sixty and back to zero again: the metabolic life of B cells. Curr. Opin. Immunol. 57, 1-7 (2019).
58. Victora, G. D. et al. Identification of human germinal center light and dark zone cells and their relationship to human B-cell lymphomas. Blood 120, 2240-2248 (2012).
59. Hashmi, A. A. et al. Ki67 Proliferation Index in Germinal and Non-Germinal Subtypes of Diffuse Large B-Cell Lymphoma. Cureus 13, e13120 (2021).
60. De Silva, N. S. & Klein, U. Dynamics of B cells in germinal centres. Nat. Rev. Immunol. 15, 137-148 (2015).
61. Klein, U. & Dalla-Favera, R. Germinal centres: role in B-cell physiology and malignancy. Nat. Rev. Immunol. 8, 22-33 (2008).
62. Akkaya, M., Kwak, K. & Pierce, S. K. B cell memory: building two walls of protection against pathogens. Nat. Rev. Immunol. 20, 229-238 (2020).
63. Beltman, J. B., Allen, C. D. C., Cyster, J. G. & de Boer, R. J. B cells within germinal centers migrate preferentially from dark to light zone. Proc. Natl. Acad. Sci. 108, 8755-8760 (2011).
64. Allen, C. D. C., Okada, T., Tang, H. L. & Cyster, J. G. Imaging of Germinal Center Selection Events During Affinity Maturation. Science 315, 528-531 (2007).
65. Gitlin, A. D., Shulman, Z. & Nussenzweig, M. C. Clonal selection in the germinal centre by regulated proliferation and hypermutation. Nature 509, 637-640 (2014).
66. Schwickert, T. A. et al. In vivo imaging of germinal centres reveals a dynamic open structure. Nature 446, 83-87 (2007).
67. Holmes, A. B. et al. Single-cell analysis of germinal-center B cells informs on lymphoma cell of origin and outcome. J. Exp. Med. 217, e20200483 (2020).
68. Morgan, D. & Tergaonkar, V. Unraveling B cell trajectories at single cell resolution. Trends Immunol. 43, 210-229 (2022).
69. Ruffin, A. T. et al. B cell signatures and tertiary lymphoid structures contribute to outcome in head and neck squamous cell carcinoma. Nat. Commun. 12, 3349 (2021).
70. Milpied, P. et al. Human germinal center transcriptional programs are de-synchronized in B cell lymphoma. Nat. Immunol. 19, 1013-1024 (2018).
71. Setty, M. et al. Characterization of cell fate probabilities in single-cell data with Palantir. Nat. Biotechnol. 37, 451-460 (2019).
72. Wang, W. et al. Single-cell transcriptomic atlas of the human endometrium during the menstrual cycle. Nat. Med. 26, 1644-1653 (2020).
73. Garcia-Alonso, L. et al. Mapping the temporal and spatial dynamics of the human endometrium in vivo and in vitro. Nat. Genet. 53, 1698-1711 (2021).
74. Schmandt, R. E., Iglesias, D. A., Co, N. N. & Lu, K. H. Understanding obesity and endometrial cancer risk: opportunities for prevention. Am. J. Obstet. Gynecol. 205, 518-525 (2011).
75. Renehan, A. G., Tyson, M., Egger, M., Heller, R. F. & Zwahlen, M. Body-mass index and incidence of cancer: a systematic review and meta-analysis of prospective observational studies. The Lancet 371, 569-578 (2008).
76. Noun, M., Akoumeh, R. & Abbas, I. Cell and Tissue Imaging by TOF-SIMS and MALDI-TOF: An Overview for Biological and Pharmaceutical Analysis. Microsc. Microanal. 28, 1-26 (2022).
77. Baranski, A. et al. MAUI (MBI Analysis User Interface)—An image processing pipeline for Multiplexed Mass Based Imaging. PLOS Comput. Biol. 17, e1008887 (2021).
78. Wulff, J. E. & Mitchell, M. W. A Comparison of Various Normalization Methods for LC/MS Metabolomics Data. Adv. Biosci. Biotechnol. 09, 339-351 (2018).
79. Wang, Y. et al. EASI-FISH for thick tissue defines lateral hypothalamus spatio-molecular organization. Cell 184, 6361-6377.e24 (2021).
80. Stringer, C., Wang, T., Michaelos, M. & Pachitariu, M. Cellpose: a generalist algorithm for cellular segmentation. http://biorxiv.org/lookup/doi/10.1101/2020.02.02.931238 (2020) doi:10.1101/2020.02.02.931238.
81. Greenwald, N. F. et al. Whole-cell segmentation of tissue images with human-level performance using large-scale data annotation and deep learning. http://biorxiv.org/lookup/doi/10.1101/2021.03.01.431313 (2021) doi:10.1101/2021.03.01.431313.

### REFERENCE LIST FOR EXAMPLES 10-17

1. Rappez, L. et al. SpaceM reveals metabolic states of single cells. Nat Methods 18, 799-805 (2021).
2. Biswas, S. K. Metabolic Reprogramming of Immune Cells in Cancer Progression. Immunity 43, 435-449 (2015).
3. Lim, A. R., Rathmell, W. K. & Rathmell, J. C. The tumor microenvironment as a metabolic barrier to effector T cells and immunotherapy. eLife 9, e55185 (2020).
4. Xiao, Z., Dai, Z. & Locasale, J. W. Metabolic landscape of the tumor microenvironment at single cell resolution. Nature Communications 10, 3763 (2019).
5. Carmona-Fontaine, C. et al. Metabolic origins of spatial organization in the tumor microenvironment. PNAS 114, 2934-2939 (2017).
6. Russell, S., Wojtkowiak, J., Neilson, A. & Gillies, R. J. Metabolic Profiling of healthy and cancerous tissues in 2D and 3D. Scientific Reports 7, 15285 (2017).
7. Ganesh, S. et al. Spatially resolved 3D metabolomic profiling in tissues. Science Advances 7, eabd0957 (2021).
8. Allam, M. et al. Spatially visualized single-cell pathology of highly multiplexed protein profiles in health and disease. Commun Biol 4, 1-16 (2021).
9. Nielsen, J. Systems Biology of Metabolism. Annual Review of Biochemistry 86, 245-275 (2017).
10. Fernie, A. R., Trethewey, R. N., Krotzky, A. J. & Willmitzer, L. Metabolite profiling: from diagnostics to systems biology. Nat Rev Mol Cell Biol 5, 763-769 (2004).
11. Sriyudthsak, K., Shiraishi, F. & Hirai, M. Y. Mathematical Modeling and Dynamic Simulation of Metabolic Reaction Systems Using Metabolome Time Series Data. Front Mol Biosci 3, 15 (2016).
12. Rae Buchberger, A., DeLaney, K., Johnson, J. & Li, L. Mass Spectrometry Imaging: A Review of Emerging Advancements and Future Insights. Anal Chem 90, 240-265 (2018).
13. Vickerman, J. C. Molecular imaging and depth profiling by mass spectrometry-SIMS, MALDI or DESI? Analyst 136, 2199-2217 (2011).
14. Eberlin, L. S. et al. Desorption Electrospray Ionization then MALDI Mass Spectrometry Imaging of Lipid and Protein Distributions in Single Tissue Sections. Anal. Chem. 83, 8366-8371 (2011).
15. Eberlin, L. S., Ifa, D. R., Wu, C. & Cooks, R. G. Three-Dimensional Vizualization of Mouse Brain by Lipid Analysis using Ambient Ionization Mass Spectrometry. Angew Chem Int Ed Engl 49, 873-876 (2010).
16. Henderson, F. et al. 3D DESI-MS lipid imaging in a xenograft model of glioblastoma: a proof of principle. Sci Rep 10, 16512 (2020).
17. Passarelli, M. K. et al. The 3D OrbiSIMS-label-free metabolic imaging with subcellular lateral resolution and high mass-resolving power. Nature Methods 14, 1175 (2017).
18. Brison, J. et al. TOF-SIMS 3D Imaging of Native and Non-Native Species within HeLa Cells. Anal. Chem. 85, 10869-10877 (2013).
19. Ahmed, A. U. An overview of inflammation: mechanism and consequences. Front. Biol. 6, 274 (2011).
20. Hotamisligil, G. S. Inflammation and metabolic disorders. Nature 444, 860-867 (2006).
21. Renner, K. et al. Metabolic Hallmarks of Tumor and Immune Cells in the Tumor Microenvironment. Front. Immunol. 8, (2017).
22. Guzik, T. J., Skiba, D. S., Touyz, R. M. & Harrison, D. G. The role of infiltrating immune cells in dysfunctional adipose tissue. Cardiovascular Research 113, 1009-1023 (2017).
23. Minguet, J., Smith, K. H. & Bramlage, P. Targeted therapies for treatment of non-small cell lung cancer-Recent advances and future perspectives. International Journal of Cancer 138, 2549-2561 (2016).
24. Kowanetz, M. et al. Differential regulation of PD-L1 expression by immune and tumor cells in NSCLC and the response to treatment with atezolizumab (anti-PD-L1). Proc Natl Acad Sci USA 115, E10119-E10126 (2018).
25. Liu, D., Jenkins, R. W. & Sullivan, R. J. Mechanisms of Resistance to Immune Checkpoint Blockade. Am J Clin Dermatol 20, 41-54 (2019).
26. Hegde, P. S. & Chen, D. S. Top 10 Challenges in Cancer Immunotherapy. Immunity 52, 17-35 (2020).
27. Mirnezami, R. et al. Chemical mapping of the colorectal cancer microenvironment via MALDI imaging mass spectrometry (MALDI-MSI) reveals novel cancer-associated field effects. Mol Oncol 8, 39-49 (2014).
28. Stringer, C., Wang, T., Michaelos, M. & Pachitariu, M. Cellpose: a generalist algorithm for cellular segmentation. Nat Methods 18, 100-106 (2021).
29. Leone, R. D. & Powell, J. D. Metabolism of immune cells in cancer. Nature Reviews Cancer 20, 516-531 (2020).
30. Guerra, L., Bonetti, L. & Brenner, D. Metabolic Modulation of Immunity: A New Concept in Cancer Immunotherapy. Cell Reports 32, 107848 (2020).
31. Li, J. et al. Lipid metabolism gene-wide profile and survival signature of lung adenocarcinoma. Lipids in Health and Disease 19, 222 (2020).
32. Fernindez, L. P., Gómez de Cedrón, M. & Ramirez de Molina, A. Alterations of Lipid Metabolism in Cancer: Implications in Prognosis and Treatment. Front. Oncol. 10, (2020).
33. Orita, H. et al. Selective inhibition of fatty acid synthase for lung cancer treatment. Clin Cancer Res 13, 7139-7145 (2007).
34. Artyomov, M. N. & Van den Bossche, J. Immunometabolism in the Single-Cell Era. Cell Metab 32, 710-725 (2020).
35. Sarode, P., Schaefer, M. B., Grimminger, F., Seeger, W. & Savai, R. Macrophage and Tumor Cell Cross-Talk Is Fundamental for Lung Tumor Progression: We Need to Talk. Front Oncol 10, (2020).
36. Zhang, D., Leal, A. S., Rous, F. A. & Liby, K. T. Profiling changes in metabolism and the immune microenvironment in lung tumorigenesis. Annals of Translational Medicine 0, 16-16 (2019).
37. Majem, B., Nadal, E. & Munoz-Pinedo, C. Exploiting metabolic vulnerabilities of Non small cell lung carcinoma. Seminars in Cell & Developmental Biology 98, 54-62 (2020).
38. Corn, K. C., Windham, M. A. & Rafat, M. Lipids in the tumor microenvironment: From cancer progression to treatment. Progress in Lipid Research 80, 101055 (2020).
39. Visca, P. et al. Fatty Acid Synthase (FAS) is a Marker of Increased Risk of Recurrence in Lung Carcinoma. Anticancer Research 24, 4169-4174 (2004).
40. van der Woude, L. L., Gorris, M. A. J., Halilovic, A., Figdor, C. G. & de Vries, I. J. M. Migrating into the Tumor: a Roadmap for T Cells. Trends in Cancer 3, 797-808 (2017).
41. Bruni, D., Angell, H. K. & Galon, J. The immune contexture and Immunoscore in cancer prognosis and therapeutic efficacy. Nature Reviews Cancer 20, 662-680 (2020).
42. Pages, F. et al. International validation of the consensus Immunoscore for the classification of colon cancer: a prognostic and accuracy study. The Lancet 391, 2128-2139 (2018).
43. De Felice, D. & Alaimo, A. Mechanosensitive Piezo Channels in Cancer: Focus on altered Calcium Signaling in Cancer Cells and in Tumor Progression. Cancers (Basel) 12, E1780 (2020).
44. Paul, C. D., Mistriotis, P. & Konstantopoulos, K. Cancer cell motility: lessons from migration in confined spaces. Nat Rev Cancer 17, 131-140 (2017).
45. Desai, S. P., Bhatia, S. N., Toner, M. & Irimia, D. Mitochondrial localization and the persistent migration of epithelial cancer cells. Biophys J 104, 2077-2088 (2013).
46. Hartmann, F. J. et al. Single-cell metabolic profiling of human cytotoxic T cells. Nat Biotechnol (2020) doi:10.1038/s41587-020-0651-8.
47. Yin, Z. et al. Targeting T cell metabolism in the tumor microenvironment: an anti-cancer therapeutic strategy. J Exp Clin Cancer Res 38, (2019).
48. Hua, X. & Thompson, C. B. Quiescent T cells: actively maintaining inactivity. Nature Immunology 2, 1097-1098 (2001).
49. Mahnke, Y. D., Brodie, T. M., Sallusto, F., Roederer, M. & Lugli, E. The who's who of T-cell differentiation: Human memory T-cell subsets. European Journal of Immunology 43, 2797-2809 (2013).
50. Lubeck, E., Coskun, A. F., Zhiyentayev, T., Ahmad, M. & Cai, L. Single-cell in situ RNA profiling by sequential hybridization I. Nature Methods 11, 360-361 (2014).
51. Coskun, A. F. & Cai, L. Dense transcript profiling in single cells by image correlation decoding I. Nature Methods 13, 657-660 (2016).
52. Coskun, A. F. et al. Nanoscopic subcellular imaging enabled by ion beam tomography. Nature Communications 12, 789 (2021).
53. Rovira-Clavé, X. et al. Subcellular localization of biomolecules and drug distribution by high-definition ion beam imaging. Nat Commun 12, 4628 (2021).
54. Chen, W. W. et al. Spectroscopic coherent Raman imaging of Caenorhabditis elegans reveals lipid particle diversity. Nat Chem Biol 16, 1087-1095 (2020).
55. Paine, M. R. L. et al. Three-Dimensional Mass Spectrometry Imaging Identifies Lipid Markers of Medulloblastoma Metastasis. Sci Rep 9, 2205 (2019).
56. Ramalingam, S. S. et al. Overall Survival with Osimertinib in Untreated, EGFR-Mutated Advanced NSCLC. New England Journal of Medicine 382, 41-50 (2020).
57. Commander, R. et al. Subpopulation targeting of pyruvate dehydrogenase and GLUT1 decouples metabolic heterogeneity during collective cancer cell invasion. Nat Commun 11, 1533 (2020).
58. Wong, A. K. H. et al. Broad auto-reactive IgM responses are common in critically ill patients, including those with COVID-19. Cell Rep Med 2, 100321 (2021).
59. Lewis, J. E. & Kemp, M. L. Integration of machine learning and genome-scale metabolic modeling identifies multi-omics biomarkers for radiation resistance. Nat Commun 12, 2700 (2021).
60. Grilo, A. L. & Mantalaris, A. A Predictive Mathematical Model of Cell Cycle, Metabolism, and Apoptosis of Monoclonal Antibody-Producing GS-NS0 Cells. Biotechnology Journal 14, 1800573 (2019).

1. A method of detecting analytes in a cell or tissue sample, the method comprising:
a) introducing into the cell or tissue sample at least one tagging moiety, wherein the tagging moieties can interact with specific proteins of interest;
b) detecting analytes and tagging moieties in the cell or tissue sample;
c) spatially detecting proteins in the cell or tissue sample; and
d) constructing a map of the analytes in the cell or tissue sample based on the data from steps b) and c).

2. The method of claim 1, wherein the analyte is a protein or a metabolite.

3. The method of claim 2, wherein the protein is a receptor.

4. (canceled)

5. The method of claim 1, wherein the at least one tagging moiety is an isotope-tagged antibody.

6. The method of claim 1, further comprising using two or more tagging moieties simultaneously, wherein each tagging moiety can interact with a different specific protein of interest.

7. The method of claim 1, wherein step b) comprises a spatially resolved three-dimensional metabolic profiling framework (3D-SMF).

8. The method of claim 7, wherein time-of-flight secondary ion mass spectrometry (TOF-SIMS) is used to perform 3D-SMF.

9. The method of claim 1, wherein step c) comprises imaging mass cytometry (IMC).

10. The method of claim 1, wherein step c) further comprises determining nuclear and cytosolic distributions of analytes.

11. The method of claim 1, wherein steps a), b), c) and d) are repeated at multiple spatial locations in the cell or tissue sample.

12. The method of claim 1, wherein steps a), b), c), and d) are repeated at multiple points in time.

13. The method of a claim 1, wherein steps b) and c) are performed sequentially.

14. The method of claim 1, wherein the cell or tissue sample comprises cancer cells, immune cells, or a tumor.

15. (canceled)

16. (canceled)

17. A microfluidic chip, comprising microchannels etched into a material, wherein the microchannels comprise a patterned mask of a tumor, wherein the patterned mask of the tumor is obtained by the method of claim 1.

18. The microfluidic chip of claim 17, wherein the material is polydimethylsiloxane.

19. The microfluidic chip of claim 17, wherein the microchannels are etched into the material by laser cutting.

20. The method of claim 17, wherein the patterning mask recapitulates a spatial organization of a tumor from a patient.

21. The microfluidic chip of claim 17, further comprising cells.

22. (canceled)

23. (canceled)

24. The microfluidic chip of claim 21, wherein the cells are cultured in the microchannels.

25. A method of monitoring an in situ model of a tumor, the method comprising:
preparing the microfluidic chip of claim 17, wherein the microfluidic chip comprises an in situ model of a tumor; and
monitoring behavior of the tumor model.

26-53. (canceled)
